(19) United States
(12) Patent Application Publication
(10) Pub. No.: US 2007/0212689 A1
(43) Pub. Date: Sep. 13, 2007

(54) Prenatal Diagnosis Using Cell-Free Fetal DNA in Amniotic Fluid

(76) Inventors: Diana W. Bianchi, Charlestown, MA (US); Paige B. Larrabee, Portland, OR (US); Kirby L. Johnson, Quincy, MA (US)

Correspondence Address: CHOATE, HALL & STEWART LLP, TWO INTERNATIONAL PLACE, BOSTON, MA, 02110, US

(21) Appl. No.: 10/577,341

(22) PCT Filed: Oct. 29, 2004
(86) PCT No.: PCT/US04/35929

§ 371 Date: Feb. 14, 2007

Related U.S. Application Data
(60) Provisional application No. 60/515,735, filed on Oct. 30, 2003.

(51) Int. Cl. C12Q 1/68

(52) U.S. Cl. 435/6

(57) ABSTRACT

The present invention relates to improved methods of prenatal diagnosis, screening, monitoring and/or testing. The inventive methods include the analysis by array-based hybridization of cell-free fetal DNA isolated from amniotic fluid. In addition to allowing the prenatal diagnosis of a variety of diseases and conditions, and the assessment of fetal characteristics such as fetal sex and chromosomal abnormalities, the new inventive methods provide substantially more information about the fetal genome in less time than it takes to perform a conventional metaphase karyotype analysis. In particular, the enhanced molecular karyotype methods provided by the present invention allow the detection of chromosomal aberrations that are not often detected prenatally such as microdeletions, microduplications and subtelomeric rearrangements.

### RELATED APPLICATION

This application claims priority to Provisional Patent Application No. 60/515,735, filed Oct. 30, 2003, which is incorporated herein by reference in its entirety.

### BACKGROUND OF THE INVENTION

Genetic disorders and congenital abnormalities (also called birth defects) occur in about 3 to 5% of all live births (A. Robinson and M. G. Linden, “Clinical Genetic Handbook”, 1993, Blackwell Scientific Publications: Boston, Mass.). Combined, genetic disorders and congenital abnormalities have been estimated to account for up to 30% of pediatric hospital admissions (C. R. Scriver et al., Can. Med. Assoc. J. 1973, 108: 1111-1115; E. W. Ling et al., Am. J. Perinatal. 1991, 8: 164-169) and to be responsible for about half of all childhood deaths in industrialized countries (R. J. Berry et al., Public Health Report, 1987, 102: 171-181; R. A. Hoekelman and I. B. Pless, Pediatrics, 1998, 82: 582-595). In the US, birth defects are the leading cause of infant mortality (R. N. Anderson et al., Month. Stat. Rep. 1997, Vol. 45, No 11, Suppl. 2, p. 55). Furthermore, genetic disorders and congenital anomalies contribute substantially to long-term disability; they are associated with enormous medical-care costs (A. Czeizel et al., Mutat. Res. 1984, 128: 73-103; Centers of Disease Control, Morb. Mortal. Weekly Rep. 1989, 38: 264-267; S. Kaplan, J. Am. Coll. Cardiol. 1991, 18: 319-320; C. Cunniff et al., Clin. Genet. 1995, 48: 17-22) and create a heavy psychological and emotional burden on those afflicted and/or their families. For these and other reasons, prenatal diagnosis has long been recognized as an essential facet of the clinical management of pregnancy itself as well as a critical step toward the detection, prevention, and, eventually, treatment of genetic disorders.

Conventional chromosome analysis methods have remained the gold standard for the prenatal exclusion of aneuploidy. Such methods are based on the selective staining of chromosomes originating from fetal cells, which results in the formation of a characteristic staining (or banding) pattern along the length of the chromosomes, allowing visualization and unambiguous identification of all the chromosomes. Examination of the karyotypes determined by these banding methods can reveal the presence of numerical and structural chromosomal abnormalities over the whole genome. Fetal cells for use in these karyotyping methods are arrested in the metaphase stage of mitosis, where the structures of the chromosomes appear most distinctly. Fetal cells are traditionally isolated from samples of amniotic fluid (obtained by amniocentesis), chorionic villi (obtained by chorionic villus sampling), or fetal blood (obtained by cordocentesis or percutaneous umbilical cord blood sampling). In addition to tissue sampling and selective staining, conventional banding methods also require cell culturing, which can take between 10 and 15 days depending on the tissue source, and preparation of high quality metaphase spreads, which is tedious, time-consuming and labor-intensive (B. Eiben et al., Am. J. Hum. Genet. 1990, 47: 656-663). Furthermore, conventional chromosome analysis methods have limited sensitivity, and their standard 450-550 band level of resolution does not allow detection of small or subtle chromosomal aberrations, such as, for example, those associated with microdeletion/microduplication syndromes.

In the past decade, the application of molecular biological techniques to conventional chromosome analysis has generated new clinical cytogenetics tools that have enhanced the spectrum of disorders that can be diagnosed prenatally. These new cytogenetics tools, which are being evaluated for their potential utility in prenatal diagnosis (I. Findlay et al., J. Assist. Preprod. Genet. 1998, 15: 266-275; A. T. A. Thein et al., Prenat. Diagn. 2000, 20: 275-280; B. Pertl et al., Mol. Hum. Reprod. 1999, 5: 1176-1179; E. Pergament et al., Prenatal. Diagn. 2000, 20: 215-230) include fluorescence in situ hybridization (or FISH) and related techniques, and quantitative fluorescence polymerase chain reactions (PCR). These techniques provide increased resolution for the elucidation of structural chromosome abnormalities that cannot be detected by conventional banding analysis, such as microdeletions and microduplications, subtle translocations, complex rearrangements involving several chromosomes or taking place in subtelomeric regions. In certain of these methods, cell culture is not required, which significantly reduces test times and labor. However, in contrast to conventional banding analysis, certain molecular cytogenetic methods such as FISH, which relies on the use of chromosome specific probes to detect chromosomal abnormalities, do not allow genome-wide screening and require at least some prior knowledge regarding the suspected chromosomal abnormality and its genomic location.

In addition to new techniques of prenatal diagnosis, new sources of fetal cells have also been explored. The discovery of intact fetal cells in the maternal circulation has excited general interest as an alternative source of fetal material samples to those obtained by invasive techniques such as amniocentesis, chorionic villus sampling, or percutaneous umbilical blood sampling. Extensive research has been done on intact fetal cells recovered from maternal blood. For example, it has been demonstrated by the Applicants that the number of circulating fetal nucleated cells is increased when the fetus is affected by trisomy 21 (D. W. Bianchi et al., Am. J. Hum. Genet. 1997, 61: 822-829, which is incorporated herein by reference in its entirety). Analysis of fetal cells isolated from maternal blood has also been shown to allow prenatal diagnosis of fetal chromosomal aneuploidies (S. Elias et al., Lancet, 1992, 340: 1033; D. W. Bianchi et al., Hum. Genet. 1992, 90: 368-370; D. Ganshirt-Ahlert et al., Am. J. Reprod. Immunol. 1993, 30: 193-200; J. L. Simpson et al., J. Am. Med. Assoc. 1993, 270: 2357-2361; F. de la Cruz et al., Fetal Diagn. Ther. 1998, 13: 380).

However, because of the scarcity of intact fetal cells in most maternal blood samples, clinical applications await further technological developments (D. W. Bianchi et al., Prenat. Diagn. 2002, 22: 609-615). Another obstacle is the probable persistence of fetal lymphocytes in the maternal circulation, resulting in “contamination” of fetal cells of interest (i.e., those originating from the current pregnancy). Although considerable progress has been made in isolation, separation and enrichment of fetal cells for analysis (J. L. Simpson and S. Elias, J. Am. Med. Assoc. 1993, 270: 2357-2361; M. C. Cheung et al., Nat. Genet. 1996, 14: 264-268; R. M. Bohmer et al., Br. J. Haematol. 1998, 103: 351-360; E. Di Naro et al., Mol. Hum. Reprod. 2000, 6: 571-574; E. Parano et al., Am. J. Med. Genet. 2001, 101: 262-267), these steps are time-consuming, labor-intensive and require expensive equipment.

In 1997, Lo and co-workers (Y. M. D. Lo et al., Lancet, 1997, 350: 485-487) demonstrated the presence of male fetal DNA sequences in the serum and plasma of pregnant women. Subsequently, this same group extended their observation by quantifying the fetal DNA in maternal plasma (Y. M. D. Lo et al., Am. J. Hum. Genet. 1998, 62: 768-775), and studying its kinetics and physiology (Y. M. D. Lo et al., Am. J. Hum. Genet. 1999, 64: 218-224). Since then, a multitude of clinical applications have been reported (B. Pertl and D. W. Bianchi, Obstet. Gynecol. 2001, 98: 483-490; Y. M. D. Lo et al., Clin. Chem. 1999, 45: 1747-1751) including the determination of fetal gender and identification of fetal rhesus D status (B. H. Faas et al., Lancet, 1998, 352: 1196; Y. M. D. Lo et al., New Engl. J. Med. 1998, 339: 1734-1738; S. Hahn et al., Ann. N.Y. Acad. Sci. 2000, 906: 148-152; X. Y. Zhong et al., Brit. J. Obstet. Gynaecol. 2000, 107: 766-769; H. Honda et al., Clin. Med. 2001, 47: 41-46; H. Honda et al., Hum. Genet. 2002, 110: 75-79). Elevated concentrations of circulating fetal DNA have been measured by real-time quantitative PCR technology in pregnancies with pre-eclampsia (Y. M. D. Lo et al., Clin. Med. 1999, 45: 184-188; T. N. Leung et al., Clin. Med. 2001, 47: 137-139; X. Y. Zhong et al., Ann. N.Y. Acad. Sci. 2001, 945: 134-180), preterm labor (T. N. Leung et al., Lancet, 1998, 352: 1904-1905), hypernemesis gravidarum (A. Sekizawa et al., Clin. Med. 2001, 47: 2164-2165), and invasive placenta (A. Sekizawa et al., Clin. Med. 2002, 48: 353-354). Similar approaches have been used to diagnose prenatal conditions such as myotonic dystrophy (P. Amicucci et al., Clin. Chem. 2000, 46: 301-302), achondroplasia (H. Saito et al., Lancet, 2000, 356: 1170), Down syndrome (Y. M. D. Lo et al., Clin. Med. 1999, 45: 1747-1751; X. Y. Zhong et al., Prenatal Diagn. 2000, 20: 795-798; L. L. Poon et al., Lancet, 2000, 356: 1819-1820), aneuploidy (C. P. Chen et al., Prenat. Diag. 2000, 20: 355-357; C. P. Chen et al., Clin. Chem. 2001, 47: 937-939), and paternally inherited cystic fibrosis (M. C. Gonzalez-Gonzalez et al., Prenatal Diagn. 2002, 22: 946-948).

Compared to the analysis of fetal cells present in maternal blood, the analysis of cell-free fetal DNA isolated from maternal plasma presents the advantage of being rapid, robust and easy to perform. In addition, the fetal DNA originates exclusively from the fetus involved in the current pregnancy. However, due to the presence of maternal DNA in the plasma, the use of cell-free fetal DNA for prenatal diagnosis is limited to paternally inherited disorders or to conditions de novo present in the fetus (i.e., resulting from mutant alleles that are distinguishable from those inherited from the mother). Therefore, it is not presently applicable to autosomal recessive disorders (D. W. Bianchi, Am. J. Hum. Genet. 1998, 62: 763-764).

Clearly, improved methods of prenatal diagnosis that allow for karyotypic analyses to be conducted more widely, more rapidly and more accurately than other cytogenetic techniques are still needed. In particular, timely, cost-effective and sensitive methodologies that can provide resolution of complex karyotypes and detection of small, subtle or cryptic chromosomal aberrations without prior knowledge of the chromosomal regions where abnormalities may be present, are highly desirable.

### SUMMARY OF THE INVENTION

The present invention provides an improved system for analyzing a fetus' genetic information. In particular, the present invention for allows the rapid determination of a “molecular karyotype” of the fetus. This molecular karyotype can provide more complete and/or more detailed information than is obtained from a standard banding method. Furthermore, the inventive molecular karyotype methods do not require cell culture, and can therefore be performed more rapidly than conventional fetal karyotypes.

In general, the present invention involves isolating cell-free fetal DNA from a sample of amniotic fluid, and determining a molecular karyotype from the DNA sample. In preferred embodiments, the molecular karyotype is determined by hybridizing a set of nucleic acid probes to the fetal DNA to assess the presence or absence of selected sequences. It will often be desirable to perform such hybridization on or by means of an array. In certain preferred embodiments, the collection of probes will detect representative sequences across the genome, so that overall genome integrity can be assessed. Alternatively or additionally, preferred probe sets may include specific probes that detect known mutations or alleles associated either with a disease or condition or with a selected physical or personal attribute.

Preferred methods of the invention allow simultaneous screening over the entire genome and exhibit a sensitivity and a resolution high enough for the detection and identification of small, subtle and/or cryptic chromosomal abnormalities (such as microdeletions, microduplications, and subtelomeric rearrangements) without prior knowledge regarding suspected chromosomal aberrations and their genomic location. With these important advantages, the methods of the invention may be expected to replace conventional molecular cytogenetics techniques in the future.

In one aspect, the present invention provides methods of prenatal diagnosis, which comprise steps of: providing a sample of amniotic fluid fetal DNA; analyzing the amniotic fluid fetal DNA by hybridization to obtain fetal genomic information; and based on the fetal genomic information obtained, providing a prenatal diagnosis.

In certain embodiments, the amniotic fluid fetal DNA is obtained by: providing a sample of amniotic fluid obtained from a pregnant woman; removing cell populations from the sample of amniotic fluid to obtain a remaining amniotic material; and treating the remaining amniotic material such that cell-free fetal DNA present in the remaining material is extracted and made available for analysis, resulting in amniotic fluid fetal DNA.

In certain embodiments, substantially call cell populations are removed from the sample of amniotic fluid and the amniotic fluid fetal DNA consists essentially of cell-free fetal DNA. In other embodiments, the remaining amniotic material includes some cells and the amniotic fluid fetal DNA comprises cell-free fetal DNA and DNA originating from the cells present in the remaining amniotic material. Preferably, however, no cellular expansion is performed, so the extracted amniotic fluid fetal DNA does no include DNA from expanded cells. In certain embodiments, the remaining amniotic material is frozen and stored under suitable storage conditions for a certain period of time before being submitted to DNA extraction. At the time of analysis, the frozen sample is thawed before treatment. Any remaining cell populations may be removed after thawing of the frozen material and prior to the DNA extraction step.

In certain embodiments, analyzing the amniotic fluid fetal DNA by hybridization to obtain fetal genomic information comprises using an array, such as, for example, a cDNA array, an oligonucleotide array, or a SNP array. In other embodiments, analyzing the amniotic fluid DNA is performed using array-based comparative genomic hybridization.

In certain embodiments, the extracted amniotic fluid fetal DNA is amplified, for example by PCR, before being analyzed. This amplification step may be particularly useful when only a small amount of amniotic fluid fetal DNA is available for analysis. Certain embodiments of the invention, however, do not include amplification.

In other embodiments, the extracted fetal DNA may be labeled with a detectable agent or moiety before analysis by array-based comparative genomic hybridization. A detectable agent may comprise a fluorescent label. Suitable fluorescent labels for use in the practice of the methods of the invention may comprise fluorescent dyes such as, for example, Cy-3™, Cy-5™, Texas red, FITC, Spectrum Red™, Spectrum Green™, phycoerythrin, a rhodamine, a fluorescein, a fluorescein isothiocyanine, a carbocyanine, a merocyanine, a styryl dye, an oxonol dye, a BODIPY dye, or equivalents, analogues, derivatives and combinations of these compounds. Alternatively, a detectable agent may comprise a hapten. Suitable haptens include, for example, biotin and dioxigenin.

Fetal DNA labeling may be carried out by any of a variety of methods. In certain embodiments, labeling of amniotic fluid fetal DNA with a detectable agent is performed by random priming, nick translation, PCR or tailing with terminal transferase.

In certain embodiments, fetal genomic information obtained by analysis of amniotic fluid fetal DNA by hybridization comprises chromosomal abnormalities and genome copy number changes at multiple genomic loci.

The methods of the invention include providing a prenatal diagnosis based on the fetal genomic information obtained. In certain embodiments, providing a prenatal diagnosis comprises determining the sex of the fetus carried by the pregnant woman. In other embodiments, providing a prenatal diagnosis comprises detecting and identifying a chromosomal abnormality. In still other embodiments, providing a prenatal diagnosis comprises identifying a disease or condition associated with a chromosomal abnormality.

In certain embodiments, the methods of the invention are performed when the fetus carried by the pregnant woman is suspected of having a chromosomal abnormality or when the fetus is suspected of having a disease or condition associated with a chromosomal abnormality. In other embodiments, the methods of the invention are performed when the pregnant woman is 35 or over 35 years old.

Chromosomal abnormalities that can be detected and identified by the methods of the invention include gain and loss of genetic material. A chromosomal abnormality may be an extra individual chromosome, a missing individual chromosome, an extra portion of a chromosome, a missing portion of a chromosome, a ring, a break, a chromosomal rearrangement or any combination of these chromosomal abnormalities. A chromosomal rearrangement may be a translocation, an inversion, a duplication, a deletion, an addition, or any combination thereof.

In certain embodiments, the chromosomal abnormality that is detected and identified by the methods of the invention, is not detectable by standard G-banding analysis or by conventional metaphase CGH. In other embodiments, the chromosomal abnormality that is detected and identified by the methods of the invention is a microdeletion, a microduplication or a subtelomeric rearrangement.

In certain embodiments, the chromosomal abnormality is an extra chromosome 21, a missing chromosome 21, an extra portion of chromosome 21, a missing portion of chromosome 21 or a rearrangement of chromosome 21.

In other embodiments, the chromosomal abnormality is an extra chromosome 13, 18, X or Y, a chromosomal aberration involving chromosome 1, a deletion of chromosome portion 1q21, a deletion of chromosome portion 4p16, a chromosomal aberration involving chromosome 4, a deletion on chromosome 5, a chromosomal aberration involving chromosome 7, a deletion of chromosome portion 7q11.23, a chromosomal aberration involving chromosome 8, a translocation involving chromosome 9 and chromosome 22, a chromosomal aberration involving chromosome 10, a chromosomal aberration involving chromosome 11, a deletion of chromosome portion 13q14, a deletion of chromosome portion 15q11-q13, a deletion of chromosome portion 15q21.1, a deletion of chromosome portion 16p13.3, a deletion of chromosome portion 17p11.2, a deletion of chromosome portion 17p13.3, a chromosomal aberration involving chromosome 19, a deletion of chromosome portion 22q11, and a chromosomal aberration involving chromosome X.

In certain embodiments, the disease or condition associated with a chromosomal abnormality is an aneuploidy, such as, for example, Down syndrome (also called trisomy 21), Patau syndrome (also called trisomy 13), Edward syndrome (also called trisomy 18), Turner syndrome, Klinefelter syndrome and XYY disease.

In other embodiments, the disease or condition associated with a chromosomal abnormality is an X-linked disorder, such as, Hemophilia A, Duchenne muscular dystrophy, Lesch-Nyhan syndrome, severe combined immunodeficiency, and Fragile X syndrome.

In still other embodiments, the disease or condition identified by the methods of the invention is associated with a chromosomal abnormality that is not detectable by standard G-banding analysis or by conventional metaphase CGH, such as, for example, a microdeletion, a microduplication or a subtelomeric rearrangement. The disease or condition may be a microdeletion/microduplication syndrome, such as Prader-Willi syndrome, Angelman syndrome, DiGeorge syndrome, Smith-Magenis syndrome, Rubinstein-Taybi syndrome, Miller-Dieker syndrome, Williams syndrome, and Charcot-Marie-Tooth syndrome, or a disorder selected from the group consisting of Cri du Chat syndrome, Retinoblastoma, Wolf-Hirschhorn syndrome, Wilms tumor, spinobulbar muscular atrophy, cystic fibrosis, Gaucher disease, Marfan syndrome and sickle cell anemia.

In another aspect, the present invention provides methods of prenatal diagnosis performed by analyzing amniotic fluid fetal DNA by array-based comparative genomic hybridization. The inventive methods comprise steps of: providing a test sample of amniotic fluid fetal DNA, wherein the test sample includes a plurality of nucleic acid segments comprising a substantially complete first genome with an unknown karyotype and labeled with a first detectable agent; providing a reference sample of control genomic DNA, wherein the reference sample includes a plurality of nucleic acid segments comprising a substantially complete second genome with a known karyotype and labeled with a second detectable agent; providing an array comprising a plurality of genetic probes, wherein each genetic probe is immobilized to a discrete spot on a substrate surface to form the array and wherein together the genetic probes comprise a substantially complete third genome or a subset of a third genome; contacting the array simultaneously with the test sample and reference sample under conditions wherein the nucleic acid segments in the samples can specifically hybridize to the genetic probes on the array; determining the binding of the individual nucleic acids of the test sample and reference sample to the individual genetic probes immobilized on the array to obtain a relative binding pattern; and based on the relative binding pattern obtained, providing a prenatal diagnosis.

In certain embodiments, the nucleic acid segments of the test sample and reference sample are labeled with a detectable agent using such methods as random priming, nick translation, PCR or tailing with terminal transferase.

In other embodiments, the first detectable agent comprises a first fluorescent label and the second detectable agent comprises a second fluorescent label. Preferably, the first and second fluorescent labels produce a dual-color fluorescence upon excitation. For example, the first and second fluorescent labels are Cy-3™ and Cy-5™, respectively; or Cy-5™ and Cy-3™, respectively. Alternatively, the first and second fluorescent labels are Spectrum Red™ and Spectrum Green™, respectively; or Spectrum Green™ and Spectrum Red™, respectively.

In certain embodiments, the hybridization capacity of high copy number repeat sequences present in the nucleic acids of the test and reference samples is suppressed. For example, the hybridization capacity of the repetitive sequences is suppressed by adding to the test and reference samples unlabeled blocking nucleic acids before the contacting step. Preferably, an excess of unlabeled blocking nucleic acids is added to the test and reference samples. In certain preferred embodiments, the unlabeled blocking nucleic acids are Human Cot-1 DNA.

In other preferred embodiments, the amniotic fluid fetal DNA to be used in the inventive methods of prenatal diagnosis is obtained by: providing a sample of amniotic fluid obtained from a pregnant woman; removing cell populations from the sample of amniotic fluid to obtain a remaining amniotic material; and treating this remaining material such that cell-free fetal DNA present in the remaining amniotic material is extracted and made available for analysis, resulting in amniotic fluid fetal DNA. In certain embodiments, substantially all cell populations are removed from the sample of amniotic fluid, and the treating step leads to amniotic fluid fetal DNA, which consists essentially of cell-free fetal DNA. In other embodiments, the remaining amniotic material comprises some cells and the treating step leads to amniotic fluid fetal DNA, which comprises cell-free fetal DNA and DNA originating from these cell populations. As described above, the remaining amniotic material may be frozen, stored under suitable storage conditions for a certain period of time before being thawed and submitted to the DNA extraction treatment and analysis steps. Any cell populations still present in the amniotic material may be removed after thawing of the frozen sample and prior to the extraction step.

As described above, the amniotic fluid fetal DNA may be amplified, for example by PCR, before analysis. Fetal DNA may also be labeled with a detectable agent using such methods as random priming, nick translation, PCR or tailing with terminal transferase.

In certain embodiments, the karyotype of the second genome has been determined by G-banding analysis, metaphase CGH, FISH or SKY.

In certain embodiments, determining the binding of the individual nucleic acids of the test and reference samples to the individual genetic probes immobilized on the array to obtain a relative binding pattern includes: measuring the intensity of the signals produced by the first detectable agent and second detectable agent at each discrete spot on the array; and determining the ratio of the intensities of the signals for each spot on the array.

In certain preferred embodiments, determining the binding of the individual nucleic acids of the test and reference samples to the individual genetic probes immobilized on the array to obtain a relative binding pattern includes: using a computer-assisted imaging system capable of acquiring multicolor fluorescence images to obtain a fluorescence image of the array after hybridization; and using a computer-assisted image analysis system to analyze the fluorescence image obtained, to interpret data imaged from the array and to display results as genome copy number ratios as a function of genomic locus in the third genome.

In certain embodiments, the methods of the invention are used to determine the sex of the fetus carried by the pregnant woman, to detect and identify a chromosomal abnormality, or to identify a disease or condition associated with a chromosomal abnormality. The chromosomal abnormalities that can be detected by the inventive methods, and the diseases or conditions associated with chromosomal abnormalities that can be identified by these methods are as listed above.

In certain embodiments, analysis of amniotic fluid fetal DNA by array-based comparative genomic hybridization according to the methods of the invention is performed when the fetus carried by the pregnant woman is suspected of having a chromosomal abnormality or when the fetus is suspected of having a disease or condition associated with a chromosomal abnormality. In other embodiments, analysis of amniotic fluid fetal DNA by array-based comparative genomic hybridization according to the methods of the invention is performed when the pregnant woman is 35 or over 35 years old.

In another aspect, the invention provides methods of testing amniotic fluid fetal DNA by array-based comparative genomic hybridization comprising steps of: providing a test sample of amniotic fluid fetal DNA, wherein the test sample includes a plurality of nucleic acid segments comprising a substantially complete first genome with a chromosomal micro-abnormality and labeled with a first detectable agent; providing a reference sample of control genomic DNA, wherein the reference sample includes a plurality of nucleic acid segments comprising a substantially complete second genome with a known karyotype and labeled with a second detectable agent; providing an array comprising a plurality of genetic probes, wherein each genetic probe is immobilized to a discrete spot on a substrate surface to form the array and wherein together the genetic probes comprise a substantially complete third genome or a subset of a third genome; contacting the array simultaneously with the test sample and reference sample under conditions wherein the nucleic acid segments in the samples can specifically hybridize to the genetic probes immobilized on the array; using a computer-assisted imaging system capable of acquiring multicolor fluorescence images to obtain a fluorescence image of the array after hybridization; using a computer-assisted image analysis system to analyze the fluorescence image obtained, to interpret data imaged from the array and to display results as genome copy number ratios as a function of genomic locus in the third genome; determining the karyotype of the first genome by FISH analysis; and comparing the results displayed as genome copy number ratios to the karyotype of the first genome determined by FISH.

In certain embodiments, comparing the results displayed as genome copy number ratios to the karyotype of the first genome determined by FISH includes: evaluating the degree of consistency between the results displayed as genomic copy number ratios and the karyotype of the first genome determined by FISH.

In other embodiments, comparing the results displayed as genome copy number ratios to the karyotype of the first genome determined by FISH includes: comparing the sensitivity of detection of the chromosomal micro-abnormality by FISH and by array-based comparative genomic hybridization. In still other embodiments, comparing the results displayed as genome copy number ratios to the karyotype of the first genome determined by FISH includes: comparing the selectivity of detection of the chromosomal micro-abnormality by FISH and by array-based comparative genomic hybridization.

In other embodiments, the methods of the invention further comprise cataloguing the degree of consistency, the sensitivity of detection and the selectivity of detection as a function of chromosomal micro-abnormality present in the first genome.

In certain embodiments, the chromosomal micro-abnormality is selected from the group consisting of a microdeletion, a microduplication and a subtelomeric rearrangement. In other embodiments, the chromosomal micro-abnormality is selected from the group consisting of a deletion of chromosomal portion 1q22, a deletion of chromosome portion 7q11.23, a deletion of chromosome portion 8q21, a deletion of chromosome portion 10q21.1-q22.1, a deletion of chromosome portion 15q11-q13, a deletion of chromosome portion 16p13.3, a deletion of chromosome portion 17p11.2, a deletion of chromosome portion 17p13.3, a deletion of chromosome portion 19q13.1-q13.2, and a deletion of chromosome portion 22q11.2.

In certain embodiments, the nucleic acid segments of the test sample and reference sample to be used in the inventive methods of testing are labeled with a detectable agent using such methods as random priming, nick translation, PCR or tailing with terminal transferase.

In other embodiments, the first detectable agent and second detectable agents are Cy-3™ and Cy-5™, or Spectrum Red™ and Spectrum Green™.

In certain embodiments, the hybridization capacity of high copy number repeat sequences present in the nucleic acids of the test sample and reference sample is suppressed by adding an excess of unlabeled blocking nucleic acids, such as Human Cot-1 DNA, to the test and reference samples before the contacting step.

In preferred embodiments, the amniotic fluid fetal DNA has been obtained as described above. The amniotic fluid fetal DNA obtained by isolation from a sample of amniotic fluid may be amplified, for example by PCR, before analysis, as described above.

In certain embodiments, the karyotype of the second genome has been determined by G-banding analysis, metaphase CGH, FISH or SKY.

In another aspect, the invention provides methods for identifying a chromosomal abnormality by analyzing amniotic fluid fetal DNA by array-based comparative genomic hybridization. The inventive methods comprise steps of: providing a test sample of amniotic fluid fetal DNA, wherein the fetal DNA originates from a fetus determined to have multiple congenital anomalies by sonographic examination, and wherein the test sample includes a plurality of nucleic acid segments comprising a substantially complete first genome with a normal karyotype and labeled with a first detectable agent; providing a reference sample of control amniotic fluid fetal DNA, wherein the fetal DNA originates from a fetus determined to have no congenital anomalies by sonographic examination, and wherein the reference sample includes a plurality of nucleic acid segments comprising a substantially complete second genome with a normal karyotype and labeled with a second detectable agent; providing an array comprising a plurality of genetic probes, wherein each genetic probe is immobilized to a discrete spot on a substrate surface to form the array and wherein together the genetic probes comprise a substantially complete third genome or a subset of a third genome; contacting the array simultaneously with the test sample and reference sample under conditions wherein the nucleic acid segments in the samples can specifically hybridize to the genetic probes immobilized on the array; using a computer-assisted imaging system capable of acquiring multicolor fluorescence images to obtain a fluorescence image of the array after hybridization; using a computer-assisted image analysis system to analyze the fluorescence image obtained, to interpret data imaged from the array and to display results as genome copy number ratios as a function of genomic locus in the third genome; and analyzing the results displayed to detect and identify any chromosomal abnormality present.

In certain embodiments, the karyotype of the first genome has been determined using a standard metaphase chromosome analysis with a 550 band level of resolution. In preferred embodiments, the chromosomal abnormality present is one that is not detectable by standard G-banding analysis or by metaphase CGH. For example, the chromosomal abnormality is a micro-rearrangement such as a microaddition, a microdeletion, a microduplication, a microinversion, a microtranslocation, a subtelomeric rearrangement, or any combination of these.

In preferred embodiments, the amniotic fluid fetal DNA of the test sample and the control amniotic fluid fetal DNA of the reference sample have been obtained by isolation from two different samples of amniotic fluid as described above. In certain embodiments, the test and reference samples are matched for fetal gender, site of sample acquisition, gestational age, and storage time.

In certain embodiments, the nucleic acid segments of the test sample and reference sample are labeled with a detectable agent using such methods as random priming, nick translation, PCR or tailing with terminal transferase. In other embodiments, the first detectable agent and second detectable agents are Cy-3™ and Cy-5™, or Spectrum Red™ and Spectrum Green™.

In certain embodiments, the hybridization capacity of high copy number repeat sequences present in the nucleic acids of the test sample and reference sample is suppressed by adding an excess of unlabeled blocking nucleic acids, such as Human Cot-1 DNA, to the test and reference samples before the contacting step.

In another aspect, the present invention provides kits containing the following components: materials to extract cell-free fetal DNA from a sample of amniotic fluid obtained from a pregnant woman; an array comprising a plurality of genetic probes, wherein each genetic probe is immobilized to a discrete spot on a substrate surface to form the array and wherein together the genetic probes comprise a substantially complete genome or a subset of a genome; and instructions for using the array according to the methods of the invention.

The inventive kits may optionally also contain materials to label a first sample of DNA with a first detectable agent and a second sample of DNA with a second detectable agent. Preferably, when the inventive kits comprise materials to label samples with detectable agents, the first and second detectable agents comprise fluorescent labels that produce a dual-color fluorescence upon excitation. For example, an inventive kit may contain materials to differentially label two samples of DNA with Cy-3™ and Cy-5™, or with Spectrum Red™ and Spectrum Green™.

The inventive kits may, additionally, also contain a reference sample of control genomic DNA with a known karyotype. In certain embodiments, the genome of the reference sample is karyotypically normal. In other embodiments, the genome of the reference sample is karyotypically abnormal. For example, it exhibits a chromosomal abnormality such as an extra individual chromosome, a missing individual chromosome, an extra portion of a chromosome, a missing portion of a chromosome, a ring, a break, a translocation, an inversion, a duplication, a deletion, an addition, or any combination of those. For example, an inventive kit may contain one reference sample of control DNA with a normal, female karyotype, another reference sample of control DNA with a normal, male karyotype and optionally a third reference sample of control DNA with a known chromosomal abnormality.

In certain embodiments, the inventive kits contain hybridization and wash buffers.

In other embodiments, the inventive kits contain unlabeled blocking nucleic acids such as Human Cot-1 DNA.

### BRIEF DESCRIPTION OF THE DRAWING

FIG. 1 presents a picture of an agarose gel (2% agarose/ethidium bromide stained), which shows that the samples of cell-free amniotic DNA labeled with Cy-3™ and the samples of reference male DNA and reference female DNA labeled with Cy-5™ are uniformly amplified and labeled. Lanes 1 to 8 contain the four cell-free amniotic DNA samples (each sample was loaded twice in consecutive lanes). The controls are: Cy-3™, Cy-5™, reference male DNA and reference female DNA, which were loaded in lane 9, lane 10, lanes 11 to 15 and lanes 16 to 20, respectively. A molecular weight marker was loaded between lane 10 and lane 11.

FIG. 2 shows data of an array-based comparative genomic hybridization experiment analyzed by the GenoSensor™ software. Ten out of eleven sex markers were detected with a statistical significance of <0.01, which equals to 91% analytical sensitivity. These data were obtained with no special assay optimization for the sample type.

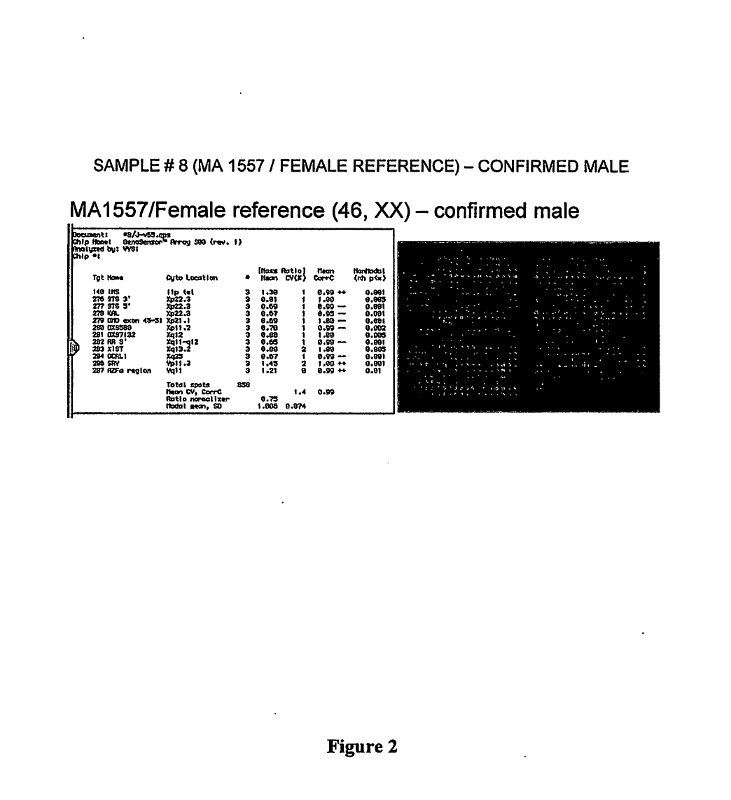

FIG. 3 shows data obtained by array-based comparative genomic hybridization experiments. Data representing chromosomes 21, X and Y are shown for each microarray hybridized with cell-free fetal DNA extracted from amniotic fluid. The results are reported as T/R (i.e., target DNA to reference DNA (euploid female reference)) ratio of fluorescence intensities (background corrected and normalized). Markers with significantly increased copy numbers (>1.2) are shown in medium grey and markers with significantly decreased copy numbers (<0.8) are shown in dark grey. Significant P-values are shown in light grey*. All male samples were compared to female reference DNA. Female 1 was compared to female reference DNA. Females 2, 3 and 4 were compared to male reference DNA. Male 5 sample was uninformative. Male 11 has known trisomy 21. (* P values <0.005 represented by 1, shown in light grey; p-values >0.005 represented by 0. Exceptions are samples: Male 9, 10 and Female 2, 3, which had significant p-values set at <0.001. Male 11 (trisomy 21) had P-values <0.05 shown as absolute numbers for chromosome 21 markers only).

FIG. 4 shows graphical data representation of array-based comparative genomic hybridization experiments. Part A and Part B present the results obtained for samples identified as female and male, respectively. The reference DNA sample used in both experiments was female.

FIG. 5 shows microarray data from two euploid and four aneuploid cell-free fetal DNA from amniotic fluid samples. Data show the expected ratio differenced for clones from chromosomes X, Y, and 21, when sample genomes are compared with a normal female genome. Samples are labeled by sex and number, followed by the karyotype of the reference DNA used for hybridization. All samples were hybridized with normal female reference DNA. Female 1 had monosomy X (Turner syndrome), Female 2 and males 3 and 4 had trisomy 21. A subset of GenoSensor Array 300 clones (Vysis), including markers on chromosomes 21, X, and Y, is shown for each array results. T/R=target DNA to reference euploid DNA ratio of Cyanine 3 (test) and Cyanine 5 (reference) fluorescent intensities (background corrected and normalized). Markers with increased copy numbers (>1.2) are highlighted in black, and markers with decreased copy numbers (<0.8) are highlighted in gray. Copy number changes with P values of <0.01 are considered significant and are underlined and shown in bold.

FIG. 6 shows a comparison of data obtained for four euploid cell-free fetal DNA from amniotic fluid samples, each hybridized separately with male and female reference DNA. Data show the expected ratio differences for clones from chromosomes X, Y, and 21, when sample genomes are compared with both a normal male genome and a normal female genome. Samples are labeled by sex and number, followed by the karyotype of the reference DNA used for hybridization. A subset of GenoSensor Array 300 (Vysis) clones, including markers on chromosomes 21, X, and Y, is shown for each array result. T/R=target DNA to reference euploid DNA ratio of fluorescent intensities (background corrected and normalized). Markers with increased copy numbers (>1.2) are highlighted in black, and markers with decreased copy numbers (<0.8) are highlighted in gray. Copy number changes with P values of <0.01 are considered significant and are underlines and shown in bold.

FIG. 7 shows a comparison of data obtained for seven euploid cell-free fetal DNA from amniotic fluid samples and their corresponding amniocyte (cellular) DNA. Data show the expected ratio differences for clones from chromosomes X, Y, and 21, when genomes from cell-free fetal DNA and genomes from cellular DNA are compared with a normal female genome. Cell-free fetal DNA hybridized to the arrays nearly as well as did the DNA extracted from whole cells. Samples are labelled by sex and number, followed by the karyotype of the reference DNA used for hybridization. All samples were hybridized with normal female reference DNA. A subset of GenoSensor Array 300 (Vysis) clones, including markers on chromosomes 21, X, and Y, is shown for each array result. T/R=target DNA to reference euploid DNA ratio of fluorescent intensities (background corrected and normalized). Markers with increased copy numbers (>1.2) are highlighted in black, and markers with decreased copy numbers (<0.8) are highlighted in gray. Copy number changes with P values <0.01 are considered significant and are underlined and shown in bold.

### DEFINITIONS

Unless otherwise stated, all technical and scientific terms used herein have the meaning commonly understood by a person skilled in the art to which this invention belongs. The following terms have the meaning ascribed to them unless specified otherwise.

As used herein, the term “prenatal diagnosis” refers to the determination of the health and conditions of a fetus, including the detection of defects or abnormalities as well as the diagnosis of diseases. A variety of non-invasive and invasive techniques are available for prenatal diagnosis. Each of them can be used only during specific time periods of the pregnancy for greatest utility. These techniques include, for example, ultrasonography, maternal serum screening, amniocentesis, and chorionic villus sampling (or CVS). The methods of prenatal diagnosis of the present invention include the analysis by array-based hybridization of cell-free fetal DNA isolated from amniotic fluid. The inventive methods of prenatal diagnosis allow for determination of fetal characteristics such as fetal sex and chromosomal abnormality, and for identification of fetal diseases or conditions.

The terms “sonographic examination”, “ultrasonographic examination”, and “ultrasound examination” are used herein interchangeably. They refer to a clinical non-invasive procedure in which high frequency sound waves are used to produce visible images from the pattern of echos made by different tissues and organs of the fetus. A sonographic examination may be used to determine the size and position of the fetus, the size and position of the placenta, the amount of amniotic fluid, and the appearance of fetal anatomy. Ultrasound examinations can reveal the presence of congenital anomalies (i.e., anatomical or structural malformations that are present at birth).

The term “amniocentesis”, as used herein, refers to a prenatal test performed by inserting a long needle in the mother's lower abdomen into the amniotic cavity inside the uterus using ultrasound to guide the needle, and withdrawing a small amount of amniotic fluid. The amniotic fluid contains skin, kidney, and lung cells from the fetus. In conventional amniocentesis, these cells are grown in culture and tested for chromosomal abnormalities by determination and analysis of their karyotypes and the amniotic fluid itself can be tested for biochemical abnormalities. As discovered by the Applicants (see below), the amniotic fluid also contains cell-free fetal DNA.

The term “chromosome” has herein its art understood meaning. It refers to structures composed of very long DNA molecules (and associated proteins) that carry most of the hereditary information of an organism. Chromosomes are divided into functional units called “genes”, each of which contains the genetic code (i.e., instructions) for making a specific protein or RNA molecule. In humans, a normal body cell contains 46 chromosomes; a normal reproductive cell contains 23 chromosomes.

The terms “chromosomal abnormality”, “chromosomal aberration” and “chromosomal alteration” are used herein interchangeably. They refer to a difference (i.e., a variation) in the number of chromosomes or to a difference (i.e., a modification) in the structural organization of one or more chromosomes as compared to chromosomal number and structural organization in a karyotypically normal individual. As used herein, these terms are also meant to encompass abnormalities taking place at the gene level. The presence of an abnormal number of (i.e., either too many or too few) chromosomes is called “aneuploidy”. Examples of aneuploidy are trisomy 21 and trisomy 13. Structural chromosomal abnormalities include: deletions (e.g., absence of one or more nucleotides normally present in a gene sequence, absence of an entire gene, or missing portion of a chromosome), additions (e.g., presence of one or more nucleotides usually absent in a gene sequence, presence of extra copies of a gene (also called duplication), or presence of an extra portion of a chromosome), rings, breaks and chromosomal rearrangements. Abnormalities that involve deletions or additions of chromosomal material alter the gene balance of an organism and if they disrupt or delete active genes, they generally lead to fetal death or to serious mental and physical defects. Structural rearrangements of chromosomes result from chromosome breakage caused by damage to DNA, errors in recombination, or crossing over the maternal and paternal ends of the separated double helix during meiosis or gamete cell division. Chromosomal rearrangements may be translocations or inversions. A translocation results from a process in which genetic material is transferred from one gene to another. A translocation is balanced when two chromosomes exchange pieces without loss of genetic material, while an unbalanced translocation occurs when chromosomes either gain or lose genetic material. Translocations may involve two chromosomes or only one chromosome. Inversions are produced by a process in which two breaks occur in a chromosome and the broken segment rotates 180°, resulting in the genes being rearranged in reverse order.

As used herein, the term “chromosomal micro-abnormality” refers to a small, subtle and/or cryptic chromosomal abnormality (for example, one involving one or more nucleotides in a gene sequence, or resulting in loss or gain of a single gene copy or one taking place at a subtelomeric region).

As used herein, the terms “microdeletion”, “microaddition”, “micro-duplication”, “microrearrangment”, “microtranslocation”, “microinversion”, and “subtelomeric rearrangement” refer to chromosomal micro-abnormalities that cannot be detected or are not easily detectable by standard cytogenetic methods, such as, for example, conventional G-banding or metaphase CGH.

As used herein, the term “disease or condition associated with a chromosomal abnormality” refers to any disease, disorder, condition or defect, that is known or suspected to be caused by a chromosomal abnormality. Exemplary diseases or conditions associated with a chromosomal abnormality include, but are not limited to, trisomies (e.g., Down syndrome, Edward syndrome, Patau syndrome, Turner syndrome, Klinefelter syndrome, and XYY disease), and X-linked disorders (e.g., Duchenne muscular dystrophy, hemophilia A, certain forms of severe combined immunodeficiency, Lesch-Nyhan syndrome, and Fragile X syndrome). Additional examples of diseases or conditions associated with chromosomal abnormalities are given below and may also be found in “Harrison's Principles of Internal Medicine”, Wilson et al. (Ed.), 1991 (12^{th} Ed.), McGraw Hill: New York, N.Y., pp 24-46, which is incorporated herein by reference in its entirety.

As used herein, the term “microdeletion/microduplication syndromes” refers to a collection of genetic syndromes that are associated with small or subtle structural chromosomal aberrations, a large number of which are beyond the resolution of detection of standard cytogenetic methods. Microdeletion/microduplication syndromes include, but are not limited to: Prader-Willi syndrome, Angelman syndrome, DiGeorge syndrome, Smith-Magenis syndrome, Rubinstein-Taybi syndrome, Miller-Dieker syndrome, Williams syndrome, and Charcot-Marie-Tooth syndrome.

As used herein, the term “karyotype” refers to the particular chromosome complement of an individual or a related group of individuals, as defined by the number and morphology of the chromosomes usually in mitotic metaphase. More specifically, a karyotype includes such information as total chromosome number, copy number of individual chromosome types (e.g., the number of copies of chromosome Y) and chromosomal morphology (e.g., length, centromeric index, connectedness and the like). Examination of a karyotype allows detection and identification of chromosomal abnormalities (e.g., extra, missing, or broken chromosomes). Since certain diseases and conditions are associated with characteristic chromosomal abnormalities, analysis of a karyotype allows diagnosis of these diseases and conditions.

As used herein, the term “G (or Giemsa) banding” refers to a standard staining technique for karyotyping. G-banding (also known as G-T-G banding) involves the use of an enzyme (the protease trypsin) to degrade some of the proteins that are associated with the chromosomes and the use of a staining dye (Giemsa) that selectively binds to DNA regions rich in guanine and cytosine. This selective staining leads to the formation of a distinctive pattern of alternating dark and light bands along the length of the chromosome, that is characteristic of the individual chromosome (light bands correspond to euchromatin, which is active DNA rich in guanine and cytosine; dark bands correspond to, which is unexpressed DNA rich in adenine and thymine). This staining reveals extra and missing chromosomes, large deletions and duplications, as well as the locations of centromeres (the major constrictions in chromosomes). However less extensive or more complex rearrangements of genetic material, chromosomal origins of markers, and subtle translocations are not detectable or are difficult to identify with certainty using standard G-banding (Giemsa, Leishman's or variant). For more details on how to perform a G-banding analysis, see, for example, J. M. Scheres et al., Hum. Genet. 1982, 61: 8-11; and K. Wakui et al., J. Hum. Genet. 1999, 44: 85-90, each of which if incorporated herein by reference in its entirety.

As used herein, the term “Fluorescence In Situ Hybridization or FISH” refers to a molecular cytogenetic technique that can be used to generate karyotypes. In a FISH experiment, specifically designed fluorescent molecules are used to visualize particular genes or sections of chromosomes by fluorescence microscopy, thus allowing detection of chromosomal abnormalities. FISH on interphase nuclei (mainly from uncultured amniocytes) is an increasingly popular tool for the rapid exclusion of selected aneuploidies (see, for example, T. Bryndorf et al., Acta Obstet. Gynecol. Scand, 2000, 79: 8-14; W. Cheong Leung et al., Prenat. Diagn. 2001, 21: 327-332; 3. Pepperberg et al., Prenat. Diagn. 2001, 21: 293-301; S. Weremowicz et al., Prenat. Diagn. 2001, 21: 262-269; and R. Sawa et al., J. Obstet. Gynaecol. Res. 2001, 27: 41-47, each of which if incorporated herein by reference in its entirety).

As used herein, the term “Spectral Karyotyping or SKY”, refers to a molecular cytogenetic technique that allows for the simultaneous visualization of all human (or mouse) chromosomes in different colors, which considerably facilitates karyotype analysis. SKY involves the preparation of a library of short sequences of single-stranded DNA labeled with spectrally distinguishable fluorescent dyes. Each of the individual probes in this DNA library is complementary to a unique region of a chromosome, while together all the probes male up a collection of DNA that is complementary to all of the chromosomes within the human genome. After in situ hybridization, the measurement of defined emission spectra by spectral imaging allows for the definitive discernment of all human chromosomes in different colors and the detection of chromosomal abnormalities, such as translocations, chromosomal breakpoints, and rearrangements. For more details about the SKY technique and its use in determining karyotypes, see, for example, E. Shrock et al., Hum. Genet. 1997, 101: 255-262; I. B. Van den Veyver and B. B. Roa, Curr. Opin. Obstet. Gynecol. 1998, 10: 97-103; M. C. Phelan et al., Prenatal Diagn. 1998, 18: 1174-1180; B. R. Haddad et al., Hum. Genet. 1998, 103: 619-625; and B. Peschka et al., Prenatal. Diagn. 1999, 19: 1143-1149, each of which is incorporated herein by reference in its entirety.

The terms “comparative genomic hybridization or CGH” and “metaphase comparative genomic hybridization or metaphase CGH” are used herein interchangeably. They refer to a molecular cytogenetic technique that involves differentially labeling a test DNA and normal reference DNA with fluorescent dyes, co-hybridizing the two labeled DNA samples to normal metaphase chromosome spreads, and visualizing the two hybridized DNAs by fluorescence. The ratio of the intensity of the two fluorescent dyes along a certain chromosome or chromosomal region reflects the relative copy number (i.e., abundance) of the respective nucleic acid sequences in the two samples. A CGH analysis provides a global overview of gains and losses of genetic material throughout the whole genome. As used herein, the term “standard metaphase chromosome analysis” refers to conventional G-banding analysis or metaphase CGH.

In contrast to metaphase CGH, “array-based comparative genomic hybridization or array-based CGH” uses immobilized gene-specific nucleic acid sequences arranged as an array on a biochip or a micro-array platform. In certain embodiments, the methods of the invention include analysis by array-based comparative genomic hybridization of cell-free fetal DNA isolated from amniotic fluid.

As used herein, the term “array-based hybridization” refers to an array-based method of DNA analysis (such as, for example, array-based CGH) that provides genomic information, such as gain and loss of genetic material, chromosomal abnormalities and genome copy number changes at multiple genomic loci.

The term “array”, “micro-array”, and “biochip” are used herein interchangeably. They refer to an arrangement, on a substrate surface, of multiple nucleic acid molecules of known sequences. Each nucleic acid molecule is immobilized to a “discrete spot” (i.e., a defined location or assigned position) on the substrate surface. The term “micro-array” more specifically refers to an array that is miniaturized so as to require microscopic examination for visual evaluation. The arrays used in the methods of the invention are preferably microarrays.

The term “nucleic acid” and “nucleic acid molecule” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products.

The terms “genomic DNA” and “genomic nucleic acid” are used herein interchangeably. They refer to nucleic acid isolated from a nucleus of one or more cells, and include nucleic acid derived from (i.e., isolated from, amplified from, cloned from as well as synthetic versions of) genomic DNA. Fetal DNA isolated from amniotic fluid may be considered as genomic DNA as it was found to represent the entire genome equally.

The term “sample of DNA” (as used, for example, in “sample of amniotic fluid fetal DNA” or “sample of control genomic DNA”) refers to a sample comprising DNA or nucleic acid representative of DNA isolated from a natural source and in a form suitable for hybridization (e.g., as a soluble aqueous solution) to another nucleic acid (e.g., immobilized on an array). Samples of DNA to be used in the practice of the present invention include a plurality of nucleic acid segments (or fragments) which together cover a substantially complete genome.

The term “genetic probe”, as used in the context of the present invention, refers to a nucleic acid molecule of known sequence immobilized to a discrete spot on an array. A genetic probe has its origin in a defined region of the genome (for example a clone or several contiguous clones from a genomic library). The sequences of the genetic probes are those for which comparative copy number information is desired. A genetic probe may also be an inter-Alu or Degenerate Oligonucleotide Primer PCR product of such clones. Together all the genetic probes may cover a substantially complete genome or a defined subset of a genome. In an array-based hybridization analysis according to the methods of the invention, genetic probes are gene-specific DNA sequences to which nucleic acid fragments from a test sample of amniotic fluid fetal DNA are hybridized. Genetic probes are capable of specifically binding (or specifically hybridizing) to nucleic acid of complementary sequence through one or more types of chemical bonds, usually through hydrogen bond formation.

The term “hybridization” refers to the binding of two single stranded nucleic acids via complementary base pairing. The terms “specific hybridization” (or “specifically hybridizes to”) and “specific binding” (or “specifically binds to”) are used herein interchangeably. They refer to a process in which a nucleic acid molecule preferentially binds, duplexes, or hybridizes to a particular nucleic acid sequence under stringent conditions. In the context of the present invention, these terms more specifically refer to a process in which a nucleic acid fragment (or segment) from a test or reference sample preferentially binds to a particular genetic probe immobilized on an array and to a lesser extend, or not at all, to other arrayed genetic probes. Hybridization between two nucleic acid molecules includes minor mismatches that can be accommodated by reducing the stringency of the hybridization/wash media to achieve the desired detection of the sequence of interest.

In the context of the present invention, the term “fetal genomic information” refers to any kind of information that can be extracted from the results obtained through analysis of amniotic fluid fetal DNA by array-based hybridization. Fetal genomic information includes, for example, gain and loss of genetic material, chromosomal abnormalities and genome copy number changes or ratios at multiple genomic loci.

As used herein, the term “genomic locus” refers to a defined portion of a genome. In the methods of the invention, each genetic probe immobilized to a discrete spot on an array has a sequence that is specific to (or characteristic of) a particular genomic locus. In an array-based comparative genomic hybridization experiment, the ratio of intensity of two differentially labeled test and reference samples at a given spot on the array reflects the genome copy number ratio of the two samples at a particular genomic locus.

The term “made available for analysis” is used herein to specify that amniotic fluid fetal DNA is manipulated (e.g., amplified, labeled, cloned, fragmented, purified, and/or concentrated and resuspended in a soluble aqueous solution) such that it is in a form suitable for hybridization to another nucleic acid (e.g., immobilized on an array).

The term “Polymerase Chain Reaction or PCR” has herein its art understood meaning and refers to a technique for making multiple copies of a specific stretch of DNA or RNA. PCR can be used to test for mutations in DNA. PCR can also be used to quantify the amount of nucleic acid in a sample. PCR can also be used to sub-clone and/or to label nucleic acid molecules. Methods of performing PCR experiments are well known in the art.

The terms “labeled”, “labeled with a detectable agent”, and “labeled with a detectable moiety” are used herein interchangeably. They are used to specify that a nucleic acid molecule or individual nucleic acid segments from a sample can be visualized following binding (i.e., hybridization) to genetic probes immobilized on an array. Samples of nucleic acid segments to be used in the methods of the invention may be detectably labeled before the hybridization reaction or a detectable label may be selected that binds to the hybridization product. Preferably, the detectable agent or moiety is selected such that it generates a signal which can be measured and whose intensity is related to the amount of hybridized nucleic acids. Preferably, the detectable agent or moiety is also selected such that it generates a localized signal, thereby allowing spatial resolution of the signal from each spot on the array. Methods for labeling nucleic acid molecules are well known in the art (see below for a more detailed description of such methods). Labeled nucleic acid fragments can be prepared by incorporation of or conjugation to a label, that is directly or indirectly detectable by spectroscopic, photochemical, biochemical, immunochemical, electrical, optical, or chemical means. Suitable detectable agents include, but are not limited to: various ligands, radionuclides, fluorescent dyes, chemiluminescent agents, microparticles, enzymes, colorimetric labels, magnetic labels, and haptens. Detectable moieties can also be biological molecules such as molecular beacons and aptamer beacons.

The terms “fluorophore”, “fluorescent moiety”, “fluorescent label”, “fluorescent dye” and “fluorescent labeling moiety” are used herein interchangeably. They refer to a molecule which, in solution and upon excitation with light of appropriate wavelength, emits light back. Numerous fluorescent dyes of a wide variety of structures and characteristics are suitable for use in the practice of this invention. Similarly, methods and materials are known for fluorescently labeling nucleic acids (see, for example, R. P. Haugland, “Molecular Probes: Handbook of Fluorescent Probes and Research Chemicals 1992-1994”, 5^{th} Ed., 1994, Molecular Probes, Inc., which is incorporated herein by reference in its entirety). In choosing a fluorophore, it is preferred that the fluorescent molecule absorbs light and emits fluorescence with high efficiency (i.e., it has a high molar absorption coefficient and a high fluorescence quantum yield, respectively) and is photostable (i.e., it does not undergo significant degradation upon light excitation within the time necessary to perform the array-based hybridization analysis). Suitable fluorescent labels for use in the practice of the methods of the invention include, for example, Cy-3™, Cy-5™, Texas red, FITC, Spectrum Red™, Spectrum Green™, phycoerythrin, rhodamine, fluorescein, fluorescein isothiocyanine, carbocyanine, merocyanine, styryl dye, oxonol dye, BODIPY dye, and equivalents, analogues or derivatives of these molecules.

The term “differentially labeled” is used to specify that two samples of nucleic acid segments are labeled with a first detectable agent and a second detectable agent that produce distinguishable signals. Detectable agents that produce distinguishable signals include matched pairs of fluorescent dyes. Matched pairs of fluorescent dyes are known in the art and include, for example, rhodamine and fluorescein, Cy-3™ and Cy-5™, and Spectrum Red™ and Spectrum Green™.

The terms “Cy-3™” and “Cy-5™” refer to fluorescent cyanine dyes (i.e., 3- and 5-N,N′-diethyltetramethylindodicarbocyanine, respectively) produced by Amersham Pharmacia Biotech (Piscataway, N.J.) (see, for example, U.S. Pat. Nos. 5,047,519; 5,151,507; 5,286,486; 5,714,386; and 6,027,709). These dyes are typically incorporated into nucleic acids in the form of 5′-amino-propargyl-2′-deoxycytidine 5′-triphosphate coupled to Cy-3™ or Cy-5™.

The terms “Spectrum Red™” and “Spectrum Green™” refer to dyes commercially available from Vysis Inc. (Downers Grove, Ill.).

As used herein, the term “computer-assisted imaging system” refers to a system capable of acquiring multicolor fluorescence images that can be used to analyze a CGH-array after hybridization and to obtain a fluorescence image of the array after hybridization. A computer-assisted imaging system is composed of a hardware, which may comprise an illumination source (such as a laser), a CCD (i.e., charge coupled device) camera, a set of filters, and a computer.

As used herein, the term “computer-assisted image analysis system” refers to a system that can be used to analyze a fluorescence image of an array after hybridization, to interpret data imaged from the array and to display results of the array-based comparative genomic hybridization as genome copy number ratios as a function of genomic locus in the arrayed genome. A computer-assisted image analysis system may comprise a computer with a software for fluorescence quantitation and fluorescence ratio determination at discrete spots on arrays.

As used herein, the term “computer” is used in its broadest general contexts and incorporate all such devices. The methods of the invention can be practiced using any computer and in conjunction with any known software or methodology. The computer can further include any form of memory elements, such as dynamic random access memory, flash memory or the like, or mass storage such as magnetic disc optional storage.

### DETAILED DESCRIPTION OF CERTAIN PREFERRED EMBODIMENTS

The present invention is directed to improved strategies for prenatal diagnosis, screening, monitoring and/or testing. In particular, highly sensitive systems are described that allow for the rapid prenatal diagnosis of diseases or conditions and the assessment of fetal characteristics such as fetal sex and chromosomal abnormalities. More specifically, the present invention encompasses the recognition, by the Applicants, that amniotic fluid is a rich source of fetal nucleic acids, relates to methods comprising the use of hybridization or array-based hybridization to analyze cell-free fetal DNA isolated from amniotic fluid. The present invention provides systems that allow for identification of chromosomal abnormalities and genome copy number variations at multiple genomic loci simultaneously and without prior knowledge of the chromosomal/genomic location(s) where changes may have occurred. In addition to requiring only small amounts of amniotic fluid material, the inventive methods also have the advantage of providing substantially more information in less time than other conventional methodologies. In particular, the methods of the invention allow for detection of small, subtle and/or cryptic chromosomal abnormalities such as microdeletions, microduplications and subtelomeric rearrangements that are not detected by routine karyotyping methods.

I. Cell-Free Fetal DNA from Amniotic Fluid

In one aspect, the methods of the invention comprise analysis of cell-free fetal DNA isolated from amniotic fluid.

In many cases, only small amounts of amniotic fluid are available for study using nucleic acid-based technology. As a consequence, these methods require lengthy sample enrichment steps (such as culture of amniotic cells), resulting in long test times that may place a significant emotional burden on the prospective parents. Preliminary work carried out in the Applicants' laboratory (D. W. Bianchi et al., Clin. Chem. 2001, 47: 1867-1869, which is incorporated herein by reference in its entirety) has demonstrated that cell-free fetal DNA is present in large amounts in the amniotic fluid and that it can be isolated easily using standard procedures. Furthermore, it was found that there is 100-200 fold more fetal DNA per milliliter of fluid in the amniotic fluid compartment as compared with maternal serum and plasma. The relative abundance of fetal DNA in the amniotic fluid eliminates (or at least significantly reduces the number of) time-consuming sample enrichment steps thereby reducing the test time and labor.

Amniotic Fluid Sample

Practicing the methods of the invention involves providing a sample of amniotic fluid obtained from a pregnant woman. Amniotic fluid is generally collected using a method called amniocentesis, in which a long needle is inserted in the mother's lower abdomen into the amniotic cavity inside the uterus; and a small amount of amniotic fluid is withdrawn.

For prenatal diagnosis, most amniocenteses are performed between the 14^{th} and 20^{th} weeks of pregnancy. The most common indications for amniocentesis include: advanced maternal age (typically set, in the US, at 35 or more than 35 years at the estimated time of delivery), previous child with a birth defect or genetic disorder, parental chromosomal rearrangement, family history of late-onset disorders with genetic components, recurrent miscarriages, positive maternal serum screening test (Multiple Marker Screening) documenting increased risk of fetal neural tube defects and/or fetal chromosomal abnormality, and abnormal fetal ultrasound examination (for example, revealing signs known to be associated with fetal aneuploidy). Risks with amniocentesis are uncommon, but include fetal loss and maternal Rh sensitization. The increased risk of fetal mortality following amniocentesis is about 0.5 to 1% above what would normally be expected. Side effects to the mother include cramping, bleeding, infection and leaking of amniotic fluid following the procedure.

Amniocentesis is presently one of the clinical tests that detect the greatest variety of fetal impairments. In conventional amniocentesis procedures, fetal cells present in the amniotic fluid are isolated by centrifugation and grown in culture for chromosome analysis, biochemical analysis and molecular biological analysis. Centrifugation, which removes cell populations from the amniotic fluid, also produces a supernatant sample (herein termed “remaining amniotic material”). This sample is usually stored at −20° C. as a back-up in case of assay failure. Aliquots of this supernatant may also be used for additional assays such as determination of alpha-fetoprotein and acetyl cholinesterase levels. After a certain period of time, the frozen supernatant sample is typically discarded. The standard protocol followed by the Cytogenetics Laboratory at Tufts-New England Medical Center (Boston, Mass.), which provides samples of remaining amniotic material to the Applicants is described in detail in Example 1.

Isolation of Cell-Free Fetal DNA

Cell-free fetal DNA for use in the methods of the present invention is isolated from a sample of amniotic fluid obtained from a pregnant woman. The isolation may be carried out by any suitable method of DNA isolation or extraction.

In preferred embodiments, cell-free fetal DNA is isolated from the remaining amniotic material obtained after removal of cell populations from a sample of amniotic fluid. The cell populations may be removed from the amniotic fluid by any suitable method, for example, by centrifugation.

In certain embodiments, substantially all the cell populations are removed from the amniotic fluid, for example, by performing more than one centrifugation. In other embodiments, the remaining amniotic material includes some cell populations.

As already mentioned above, before isolation or extraction of cell-free fetal DNA, the remaining amniotic material may be frozen and stored for a certain period of time under suitable storage conditions. Fetal DNA stored at −20° C. for up to 8 years was found to be suitable for array-based hybridization experiments. Before extraction, the frozen sample is thawed at 37° C. and then mixed with a vortex. Any remaining cell populations still present in the amniotic fluid sample may be eliminated by centrifugation.

Isolating fetal DNA includes treating the remaining amniotic material such that cell-free fetal DNA present in the remaining amniotic material is extracted and made available for analysis. Any suitable isolation method that results in extracted amniotic fluid fetal DNA may be used in the practice of the invention.

Methods of DNA extraction are well known in the art. A classical DNA isolation protocol is based on extraction using organic solvents such as a mixture of phenol and chloroform, followed by precipitation with ethanol (see, for example, J. Sambrook et al., “Molecular Cloning: A Laboratory Manual”, 1989, 2^{nd} Ed., Cold

Spring Harbour Laboratory Press: New York, N.Y.). Other methods include: salting out DNA extraction (see, for example, P. Sunnucks et al., Genetics, 1996, 144: 747-756; and S. M. Aljanabi and I. Martinez, Nucl. Acids Res. 1997, 25: 4692-4693); the trimethylammonium bromide salts DNA extraction method (see, for example, S. Gustincich et al., BioTechniques, 1991, 11: 298-302) and the guanidinium thiocyanate DNA extraction method (see, for example, J. B. W. Hammond et al., Biochemistry, 1996, 240: 298-300).

There are also numerous different and versatile kits that can be used to extract DNA from bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

Typically, fetal DNA extraction is carried out on aliquots of from about 8 mL to about 15 mL of remaining amniotic material. Preferably, the extraction is carried out on an aliquot of from about 12 mL to about 15 mL of remaining amniotic material. More preferably, the extraction is carried out on an aliquot of more than 15 mL of remaining amniotic material.

When substantially all cell populations are removed from the sample of amniotic fluid, the amniotic fluid fetal DNA consists essentially of cell-free fetal DNA. When only part of all the cell populations are removed from the sample of amniotic fluid, the amniotic fetal DNA comprises cell-free fetal DNA as well as DNA originating from the cells that were still present in the remaining amniotic material. In the latter case, a larger amount of DNA is generally obtained.

DNA extractions carried out, by the Applicants, on samples of remaining amniotic material of ≧10 mL in volume, using the “Blood and Body Fluid” protocol as described by Qiagen, yielded between 8 and 900 ng of fetal DNA. Cell-free fetal DNA isolated from amniotic fluid was found to represent the whole genome equally.

Amplification of Extracted Cell-Free Fetal DNA

In certain embodiments, the amniotic fluid fetal DNA is amplified before being analyzed by hybridization. An amplification step may be particularly useful when only a small amount of amniotic fluid fetal DNA is available for analysis.

Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York, N.Y.; “Short Protocols in Molecular Biology”, F. M. Ausubel (Ed.), 2002, 5^{th} Ed., John Wiley & Sons; U.S. Pat. Nos. 4,683,195; 4,683,202 and 4,800,159). Standard nucleic acid amplification methods include: polymerase chain reaction (or PCR, see, for example, “PCR Protocols: A Guide to Methods and Applications”, M. A. Innis (Ed.), Academic Press: New York, 1990; and “PCR Strategies”, M. A. Innis (Ed.), Academic Press: New York, 1995); ligase chain reaction (or LCR, see, for example, U. Landegren et al., Science, 1988, 241: 1077-1080; and D. L. Barringer et al., Gene, 1990, 89: 117-122); transcription amplification (see, for example, D. Y. Kwoh et al., Proc. Natl. Acad. Sci. USA, 1989, 86: 1173-1177); self-sustained sequence replication (see, for example, J. C. Guatelli et al., Proc. Natl. Acad. Sci. USA, 1990, 87: 1874-1848); Q-beta replicase amplification (see, for example, J. H. Smith et al., J. Clin. Microbiol. 1997, 35: 1477-1491); automated Q-beta replicase amplification assay (see, for example, J. L. Burg et al., Mol. Cell. Probes, 1996, 10: 257-271) and other RNA polymerase mediated techniques such as, for example, nucleic acid sequence based amplification (or NASBA, see, for example, A. E. Greijer et al., J. Virol. Methods, 2001, 96: 133-147).

Amplification can also be used to quantify the amount of extracted fetal DNA (see, for example, U.S. Pat. No. 6,294,338). Alternatively or additionally, amplification using appropriate oligonucleotide primers can be used to subclone and/or to label cell-free fetal DNA prior to analysis by hybridization (see below). Suitable oligonucleotide amplification primers can easily be selected and designed by one skilled in the art.

Subsequent quantitative and/or qualitative analysis of amplified DNA can be carried out using known techniques, such as: digestion with restriction endonuclease, ultraviolet light visualization of ethidium bromide stained agarose gels; DNA sequencing, or hybridization with allele specific oligonucleotide probes (R. K. Saiki et al., Am. J. Hum. Genet. 1988, 43(suppl.): A35).

Labeling of Cell-Free Fetal DNA

In certain preferred embodiments, extracted fetal DNA is labeled with a detectable agent or moiety before being analyzed by hybridization. The role of a detectable agent is to allow visualization of hybridized nucleic acid fragments (e.g., nucleic acid fragments bound to genetic probes immobilized on an array). Preferably, the detectable agent is selected such that it generates a signal which can be measured and whose intensity is related (e.g., proportional) to the amount of labeled nucleic acids present in the sample being analyzed. In array-based hybridization methods of the invention, the detectable agent is also preferably selected such that is generates a localized signal, thereby allowing resolution of the signal from each spot on the array.

The association between the nucleic acid molecule and detectable agent can be covalent or non-covalent. Labeled nucleic acid fragments can be prepared by incorporation of or conjugation to a detectable moiety. Labels can be attached directly to the nucleic acid fragment or indirectly through a linker. Linkers or spacer arms of various lengths are known in the art and are commercially available, and can be selected such that they reduce steric hindrance, and/or confer other useful or desired properties to the resulting labeled molecules (see, for example, E. S. Mansfield et al., Mol. Cell. Probes, 1995, 9: 145-156).

Methods for labeling nucleic acid fragments are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of monoreactive cisplatin derivatives with the N7 position of guanine moieties in DNA (see, for example, R. J. Heetebrij et al., Cytogenet. Cell. Genet. 1999, 87: 47-52), psoralen-biotin, which intercalates into nucleic acids and becomes covalently bonded to the nucleotide bases upon UV irradiation (see, for example, C. Levenson et al., Methods Enzymol. 1990, 184: 577-583; and C. Pfannschmidt et al., Nucleic Acids Res. 1996, 24: 1702-1709), photoreactive azido derivatives (see, for example, C. Neves et al., Bioconjugate Chem. 2000, 11: 51-55), and DNA alkylating agents (see, for example, M. G. Sebestyen et al., Nat. Biotechnol. 1998, 16: 568-576).

Any of a wide variety of detectable agents can be used in the practice of the present invention. Suitable detectable agents include, but are not limited to: various ligands, radionuclides (such as, for example, ³²P, ³⁵S, ³H, ¹⁴C, ¹²⁵I, ¹³¹I, and the like); fluorescent dyes (for specific exemplary fluorescent dyes, see below); chemiluminescent agents (such as, for example, acridinium esters, stabilized dioxetanes and the like); microparticles (such as, for example, quantum dots, nanocrystals, phosphors and the like); enzymes (such as, for example, those used in an ELISA, i.e., horseradish peroxidase, beta-galactosidase, luciferase, alkaline phosphatase); calorimetric labels (such as, for example, dyes, colloidal gold and the like); magnetic labels (such as, for example, Dynabeads™); and biotin, dioxigenin or other haptens and proteins for which antisera or monoclonal antibodies are available.

In certain preferred embodiments, amniotic fluid fetal DNA to be analyzed by hybridization is fluorescently labeled. Numerous known fluorescent labeling moieties of a wide variety of chemical structures and physical characteristics are suitable for use in the practice of this invention. Suitable fluorescent dyes include, but are not limited to: Cy-3™, Cy-5™, Texas red, FITC, Spectrum Red™, Spectrum Green™, phycoerythrin, rhodamine, fluorescein, fluorescein isothiocyanine, carbocyanine, merocyanine, stylyl dye, oxonol dye, BODIPY dye (i.e., boron dipyrromethene difluoride fluorophore), and equivalents, analogues or derivatives of these molecules. Similarly, methods and materials are known for linking or incorporating fluorescent dyes to biomolecules such as nucleic acids (see, for example, R. P. Haugland, “Molecular Probes: Handbook of Fluorescent Probes and Research Chemicals 1992-1994”, 5^{th} Ed., 1994, Molecular Probes, Inc.). Fluorescent labeling agents as well as labeling kits are commercially available from, for example, Amersham Biosciences Inc. (Piscataway, N.J.), Molecular Probes Inc. (Eugene, Oreg.), and New England Biolabs Inc. (Berverly, Mass.).

Favorable properties of fluorescent labeling agents to be used in the practice of the invention include high molar absorption coefficient, high fluorescence quantum yield, and photostability. Preferred labeling fluorophores exhibit absorption and emission wavelengths in the visible (i.e., between 400 and 750 nm) rather than in the ultraviolet range of the spectrum (i.e., lower than 400 nm). Preferred fluorescent dyes include Cy-3™ and Cy-5™ (i.e., 3- and 5-N,N′-diethyltetramethylindo-dicarbocyanine, respectively). Cy-3™ exhibits a maximum absorption at 550 nm; emits fluorescence with a maximum at 570 nm; and its fluorescence quantum yield has been determined to be 0.04 when Cy-3™ is conjugated to a biomolecule (Amersham Biosciences Inc., Piscataway, N.J.). Cy-5™ displays absorption and emission fluorescent maxima at 649 and 670 nm, respectively, and its fluorescence quantum yield when conjugated to a biomolecule was reported to be 0.28 (Amersham Biosciences Inc., Piscataway, N.J.). To increase the stability of Cy-5™ (and therefore allow longer hybridization times as well as more intense fluorescence signals), antioxidants and free radical scavengers can be added to the hybridization mixture and/or to the hybridization/wash buffer solutions. Cy-3™ and Cy-5™ also present the advantage of forming a matched pair of fluorescent labels that are compatible with most fluorescence detection systems for array-based instruments (see below). Another preferred matched pair of fluorescent dyes comprises Spectrum Red™ and Spectrum Green™.

Detectable moieties can also be biological molecules such as molecular beacons and aptamer beacons. Molecular beacons are nucleic acid molecules carrying a fluorophore and a non-fluorescent quencher on their 5′ and 3′ ends. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and resulting in emission of a fluorescence signal. The use of molecular beacons as detectable moieties is well-known in the art (see, for example, D. L. Sokol et al., Proc. Natl. Acad. Sci. USA, 1998, 95: 11538-11543; and U.S. Pat. Nos. 6,277,581 and 6,235,504). Aptamer beacons are similar to molecular beacons except that they can adopt two or more conformations (see, for example, O. K. Kaboev et al., Nucleic Acids Res. 2000, 28: E94; R. Yamamoto et al., Genes Cells, 2000, 5: 389-396; N. Hamaguchi et al., Anal. Biochem. 2001, 294: 126-131; S. K. Poddar and C. T. Le, Mol. Cell. Probes, 2001, 15: 161-167).

A “tail” of normal or modified nucleotides can also be added to nucleic acid fragments for detectability purposes. A second hybridization with nucleic acid complementary to the tail and containing a detectable label (such as, for example, a fluorophore, an enzyme or bases that have been radioactively labeled) allows nucleic acid fragments bound to the array to be visualized (see, for example, the system commercially available from Enzo Biochem Inc., New York, N.Y.).

The selection of a particular nucleic acid labeling technique will depend on the situation and will be governed by several factors, such as the ease and cost of the labeling method, the quality of sample labeling desired, the effects of the detectable moiety on the hybridization reaction (e.g., on the rate and/or efficiency of the hybridization process), the nature of the detection system of the hybridization instrument to be used, the nature and intensity of the signal generated by the detectable label, and the like.

II. Array-Based Hybridization Analysis of Amniotic Fluid Fetal DNA

In another aspect, the present invention provides methods of prenatal diagnosis, screening, monitoring and/or testing, which include analysis of cell-free fetal DNA by array-based hybridization.

Developmental abnormalities, such as Down, Turner and Klinefelter syndromes, result from gain or loss of one copy of an individual chromosome or of a chromosomal region. Other conditions, such as DiGeorge, Prader-Willi, and Angelman syndromes, are associated with microdeletions or other subtle chromosomal abnormalities that are difficult to detect and may easily be missed using traditional karyotyping methods. Techniques that allow highly sensitive detection and mapping of chromosomal abnormality over a substantially complete portion of the genome provides more accurate methods of prenatal diagnosis as well as a unique approach for associating chromosomal aberrations with disease phenotype and for localizing and identifying critical genes.

The analysis of cell-free fetal DNA by array-based hybridization may be carried out by any suitable array-based hybridization method of DNA analysis that can provide genomic information, such as gain and loss of genetic material, chromosomal abnormalities and/or genome copy number changes at multiple genomic loci. Such methods include, but are not limited to: array-based comparative genomic hybridization and hybridization methods using arrays that contain individual base pair changes or mismatches.

Comparative Genomic Hybridization

Comparative Genomic Hybridization (or CGH) is a molecular cytogenetic technique that was developed to survey DNA copy number variations across a whole genome (A. Kallioniemi et al., Science, 1992, 258: 818-821; O. P. Kallioniemi et al., Semin. Cancer Biol. 1993, 4: 41-46; S. du Manoir et al., Hum. Genetics, 1993, 90: 590-610; S. Willadsen et al., Hum. Reprod. 1999, 14: 470-475, each of which is incorporated herein by reference in its entirety). CGH analyses compare the genetic composition of test versus reference samples and allow, for example, to determine whether a test sample of genomic DNA contains amplified or deleted or mutated nucleic acid segments as compared to a reference sample.

CGH is usually based on a combination of in situ hybridization, fluorescence microscopy and digital image analysis. Typically in a traditional metaphase CGH experiment, two genomic populations (i.e., one test sample and one reference sample of multi-megabase fragments of DNA), are differentially labeled with fluorescent dyes, co-hybridized in situ to normal metaphase chromosomes, and visualized by fluorescence. The ratio of intensity of the two different fluorescent labels along a certain chromosome or chromosomal region reflects the relative abundance (i.e., the relative copy number) of the respective nucleic acid sequences in the two samples. The reference sample can be selected to act as a negative control (i.e., a normal or wild-type genome) or as a positive control (i.e., sample known to contain a chromosomal aberration).

Metaphase CGH, with its whole-genome screening capability, is faster and less laborious than other karyotyping methods and has found a wide range of applications in clinical cytogenetics (see, for example, T. Bryndorf et al., Am. J. Hum. Genet. 1995, 57:1211-1220). However, metaphase CGH has a number of limitations that restrict its usefulness as a screening tool. For example, metaphase CGH was found to be less sensitive than PCR based-methods in detecting deletions. Most of the limitations displayed by metaphase CGH are inherent to the use of metaphase chromosomes. Indeed, the highly condensed and supercoiled organization of DNA in chromosomes prevents the detection of abnormalities involving small regions of the genome and the resolution of closely spaced aberrations. The resolution of metaphase CGH, while providing a valuable starting point for cytogenetic studies, does not allow precise location of sequences of interest. Conversely, a technique such as FISH (i.e., fluorescence in situ hybridization) exhibits a much higher resolution than metaphase CGH, but is too labor-intensive to be used on a genomic scale.

Array-Based Comparative Genomic Hybridization

An increased mapping resolution is achieved by array-based CGH. In contrast to metaphase CGH, in which the immobilized probe is a metaphase chromosome, array-based CGH uses immobilized gene-specific nucleic acid sequences arranged as an array on a biochip or a micro-array platform. The array-based CGH approach yields DNA sequence copy number information across a whole (or substantially complete) genome in a single, timely, and sensitive procedure, the resolution of which is primarily dependent upon the number, size and map positions of the DNA sequences within the array.

An array-based CGH experiment is similar to a metaphase CGH experiment. Equivalent amounts of a test sample and reference sample of DNA are differentially labeled with fluorescent dyes and co-hybridized to an array of cloned genomic DNA fragments that collectively cover a substantially complete genome or a subset of a genome. Each spot on the array contains a nucleic acid sequence that corresponds to a specific segment of the genome. Fluorescence ratios at discrete spots of the resulting labeled array reflect the competitive hybridization of sequences in the test and reference samples and provide a locus-by-locus measure of DNA copy-number variations. Therefore, array-based CGH allows genome-wide mapping of regions with DNA sequence copy number changes (i.e., gain and loss of genetic material) in a single experiment without previous knowledge of the locations of the chromosomal/genomic regions of abnormality (T. Bryndorf et al., Am. J. Hum. Genet. 1995, 57: 1211-1220; M. Schena et al., Proc. Natl. Acad. Sci. USA, 1996, 93: 10614-10619; and E. S. Lander, Nat. Genet. 1999, 21(suppl.): 3-4).

CGH has primarily found applications in cancer genetics as a rapid and accurate tool to detect gene amplifications and deletions, and to study their roles in tumor development and progression, and their response to therapy. Screening by comparative genomic hybridization of DNAs extracted from frozen specimens and cell lines from various tumor types has revealed a number of recurring chromosomal gains and losses that were undetected by traditional cytogenetic analysis.

Analysis of Amniotic Fluid Fetal DNA by Array-Based CGH

Certain methods of the invention include analyzing amniotic fluid fetal DNA by array-based comparative genomic hybridization.

More specifically, certain methods of the invention comprise steps of: providing a sample of amniotic fluid fetal DNA; analyzing the amniotic fluid fetal DNA by array-based comparative genomic hybridization to obtain fetal genomic information; and, based on the fetal genomic information obtained, providing a prenatal diagnosis.

The analyzing step in the methods of the invention can be performed using any of a variety of methods, means and variations thereof for carrying out array-based comparative genomic hybridization. Array-based CGH methods are known in the art and have been described in numerous scientific publications as well as in patents (see, for example, U.S. Pat. Nos. 5,635,351; 5,665,549; 5,721,098; 5,830,645; 5,856,097; 5,965,362; 5,976,790; 6,159,685; 6,197,501 and 6,335,167; and EP 1 134 293 and EP 1 026 260, each of which is incorporated herein by reference in its entirety).

Array-based CGH methods have been developed and used in medicine and clinical research, for example, in dermatology to map complex traits in diseases of the hair and skin (V. M. Aita et al., Exp Dermatol. 1999, 8: 439-452), in cancer genetics (H. Kashiwagi and K. Uchida, Hum. Cell. 2000, 13: 135-141); as a new strategy to identify novel ovarian genes (A. B. Tavares et al., Semin Reprod Med. 2001, 19: 167-173); in breast cancer research (D. Pinkel et al., Nat. Genet. 1998, 20: 207-211; J. R. Pollack et al., Nat. Genet. 1999, 23: 41-46; C. S. Cooper, Breast Cancer Res. 2001, 3: 158-175); in pancreatic cancer research (M. Buchholz et al., Pancreatology, 2001, 1: 581-586); as a novel approach for diagnostics and identification of genetically defined leukemia and lymphoma subgroups (P. Lichter et al., Semin. Hematol. 2000, 37: 348-357; T. R. Golub, Curr. Opin. Hematol. 2001, 8: 252-261; S. Wessendorf et al., Ann Hematol. 2001, 80(Suppl 3): B35-37); as a new research tool to identify genes that may be causally associated with metastasis (C. Khanna et al., Cancer Res. 2001, 61: 3750-3790); in dental research (W. P. Kuo et al., Oral Oncol. 2002, 38: 650-656); in pharmacogenomics (K. K. Jain, Pharmacogenomics, 2000, 1: 289-307); in renal research (M. Kurella et al., J. Ain. Soc. Nephrol. 2001, 12: 1072-1078); and in nutritional and obesity research (M. J. Moreno-Aliaga et al., Br. J. Nutr. 2001, 86: 119-122).

In the practice of the present invention, these methods as well as other methods known in the art for carrying out array-based comparative genomic hybridization may be used as described or modified such that they allow for fetal genomic information to be obtained. Fetal genomic information includes, but is not limited to: gain and loss of genetic material, chromosomal abnormalities and genome copy number changes at multiple genomic loci.

Other inventive methods of prenatal diagnosis performed by analyzing amniotic fluid fetal DNA by array-based comparative genomic hybridization comprise steps of: providing a test sample of amniotic fluid fetal DNA, wherein the test sample includes a plurality of nucleic acid segments comprising a substantially complete first genome with a unknown karyotype and labeled with a first detectable agent; providing a reference sample of control genomic DNA, wherein the reference sample includes a plurality of nucleic acid segments comprising a substantially complete second genome with a known karyotype and labeled with a second detectable agent; providing an array comprising a plurality of genetic probes, wherein each genetic probe is immobilized to a discrete spot on a substrate surface to form the array and wherein the genetic probes together comprise a substantially complete third genome or a subset of a third genome; contacting the array simultaneously with the test and reference samples under conditions wherein the nucleic acid segments in the test and reference samples can specifically hybridize to the genetic probes on the array; determining the binding of the individual nucleic acids in the test sample and reference sample to the individual genetic probes immobilized on the array to obtain a relative binding pattern; and providing a prenatal diagnosis based on the relative binding pattern obtained.

Test and Reference Samples

In the array-based CGH methods of the invention, a test sample of amniotic fluid fetal DNA is compared against a reference sample of control genomic DNA.

Preferably, amniotic fluid fetal DNA is isolated from a sample of amniotic fluid as described above. A test sample of amniotic fluid fetal DNA to be used in the methods of the invention includes a plurality of nucleic acid fragments comprising a substantially complete first genome, whose karyotype is unknown.

A reference sample of control genomic DNA to be used in the methods of the invention includes a plurality of nucleic acid fragments comprising a substantially complete second genome whose karyotype is known. In the array-based CGH methods of the invention, genomic control DNA may be selected to act as a negative control (e.g., sample with a normal or wild-type genome) or as a positive control (e.g., sample containing one or more chromosomal aberrations). The reference sample of control DNA may be isolated from an individual who has either a normal 46, XX karyotype (female euploid) or a normal 46, XY karyotype (male euploid). Alternatively, the reference sample of control genomic DNA may be isolated from an individual who has a disease or condition associated with an identified chromosomal abnormality (for example, an individual with Down syndrome). The reference sample of control DNA may, alternatively, originate from a fetus and be isolated from fetal cells circulating in the maternal plasma or serum, or present in the amniotic fluid; and its karyotype may be determined by conventional G-banding analysis, metaphase CGH, FISH or SKY (D. W. Bianchi et al., Prenatal. Diagn. 1993, 13: 293-300; D. Ganshirt-Ahlert et al., Am. J. Reprod. Immunol. 1993, 30: 2-3; J. L. Simpson et al., J. Am. Med. Assoc. 1993, 270: 2357-2361; Y. I. Zheng et al., J. Med. Genet. 1993, 30: 1051-1056). Alternatively, the sample of control DNA may originate from a fetus and be isolated from a sample of amniotic fluid as described above.

The DNA from the two genomes may be amplified, labeled, fragmented, purified, concentrated and/or otherwise modified prior to the array-based CGH analysis. Techniques for the manipulation of nucleic acids are well-known in the art (see, for example, J. Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5^{th} Ed., John Wiley & Sons, each of which is incorporated herein by reference in its entirety).

In certain preferred embodiments, in order to improve the resolution of the array-based comparative genomic hybridization analysis, the nucleic acid fragments of the test and reference samples are less than about 500 bases long, preferably less than about 200 bases long. The use of small fragments significantly increases the reliability of the detection of copy number differences or the detection of unique sequences by suppressing repetitive sequences and other background cross-hybridization.

Methods of DNA fragmentation are known in the art and include: treatment with DNase, sonication (see, for example, P. L. Deininger, Anal. Biochem. 1983, 129: 216-223), mechanical shearing, and the like (see, for example, J. Sambrook et al., “Molecular Cloning: A Laboratory Manual”, 1989, 2^{nd} Ed., Cold Spring Harbour Laboratory Press: New York, N.Y.; P. Tijssen “Hybridization with Nucleic Acid Probes—Laboratory Techniques in Biochemistry and Molecular Biology (Parts I and II)”, 1993, Elsevier Science; C. P. Ordahl et al., Nucleic Acids Res. 1976, 3: 2985-2999; P. J. Oefner et al., Nucleic Acids Res. 1996, 24: 3879-3886; Y. R. Thorstenson et al., Genome Res. 1998, 8: 848-855). Random enzymatic digestion of the DNA leads to fragments containing as low as 25 to 30 bases. Such a digestion may be carried out using DNA endonucleases (see, for example, J. E. Herrera and J. B. Chaires, J. Mol. Biol. 1994, 236: 405-411; and D. Suck, J. Mol. Recognit. 1994, 7: 65-70) or the two-based restriction endonuclease, CviJI (see, for example, M. C. Fitzgerald et al., Nucl. Acids Res. 1992, 20: 3753-3762).

Fragment size of the nucleic acid segments in the test and reference samples may be evaluated by any of a variety of techniques, such as, for example, electrophoresis (see, for example, B. A. Siles and G. B. Collier, J. Chromatogr. A, 1997, 771: 319-329) or matrix-assisted laser desorption/ionization time-of-flight mass spectrometry (see, for example, N. H. Chiu et al., Nucl. Acids Res. 2000, 28: E31).

In the practice of the methods of the invention, the test sample of amniotic fluid fetal DNA and reference sample of control genomic DNA are labeled before analysis by array-based CGH. Suitable methods of nucleic acid labeling with detectable agents have been described in detail above. To allow determination of genome copy number ratios, the two DNA samples should be differentially labeled (i.e., the first detectable agent labeling the test sample and the second detectable agent labeling the reference sample should produce distinguishable signals). Matched pairs of suitable detectable agents for use in the methods of the invention have been described below.

Prior to hybridization, the labeled nucleic acid fragments of the test and reference samples may be purified and concentrated before being resuspended in the hybridization buffer. Microcon 30 columns may be used to purify and concentrate samples in a single step. Alternatively, nucleic acids may be purified using a membrane column (such as Qiagen columns) or sephadex G50 and precipitated in the presence of ethanol.

Methods of preparation of nucleic acid samples for array-based comparative genomic hybridization experiments can easily be performed and/or modified by one skilled in the art.

Comparative Genomic Hybridization Arrays

In the methods of the invention, amniotic fluid fetal DNA is analyzed by comparative genomic hybridization using an array-based approach.

Any of a variety of arrays may be used in the practice of the present invention. Investigators can either rely on commercially available arrays or generate their own. Methods of making and using arrays are well known in the art (see, for example, S. Kern and G. M. Hampton, Biotechniques, 1997, 23:120-124; M. Schummer et al., Biotechniques, 1997, 23:1087-1092; S. Solinas-Toldo et al., Genes, Chromosomes & Cancer, 1997, 20: 399-407; M. Johnston, Curr. Biol. 1998, 8: R171-R174; D. D. Bowtell, Nature Gen. 1999, Supp. 21:25-32; S. J. Watson and H. Akil, Biol Psychiatry. 1999, 45: 533-543; W. M. Freeman et al., Biotechniques. 2000, 29: 1042-1046 and 1048-1055; D. J. Lockhart and E. A. Winzeler, Nature, 2000, 405: 827-836; M. Cuzin, Transfus. Clin. Biol. 2001, 8:291-296, P. P. Zarrinkar et al., Genome Res. 2001, 11: 1256-1261; M. Gabig and G. Wegrzyn, Acta Biochim. Pol. 2001, 48: 615-622; and V. G. Cheung et al., Nature, 2001, 40: 953-958; see also, for example, U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893, each of which is incorporated herein by reference in its entirety).

Arrays comprise a plurality of genetic probes immobilized to discrete spots (i.e., defined locations or assigned positions) on a substrate surface. Substrate surfaces for use in the present invention can be made of any of a variety of rigid, semi-rigid or flexible materials that allow direct or indirect attachment (i.e., immobilization) of genetic probes to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may preferably be used (see, for example, U.S. Pat. No. 6,063,338).

The presence of reactive functional chemical groups (such as, for example, hydroxyl, carboxyl, amino groups and the like) on the material can be exploited to directly or indirectly attach genetic probes to the substrate surface. Methods for immobilizing genetic probes to substrate surfaces to form an array are well-known in the art.

More than one copy of each genetic probe may be spotted on the array (for example, in duplicate or in triplicate). This arrangement may, for example, allow assessment of the reproducibility of the results obtained (see below). Related genetic probes may also be grouped in probe elements on an array. For example, a probe element may include a plurality of related genetic probes of different lengths but comprising substantially the same sequence. Alternatively, a probe element may include a plurality of related genetic probes that are fragments of different lengths resulting from digestion of more than one copy of a cloned piece of DNA. An array may contain a plurality of probe elements. Probe elements on an array may be arranged on the substrate surface at different densities.

Array-immobilized genetic probes may be nucleic acids that contain sequences from genes (e.g., from a genomic library), including, for example, sequences that collectively cover a substantially complete genome or a subset of a genome. The sequences of the genetic probes are those for which comparative copy number information is desired. For example, to obtain DNA sequence copy number information across an entire genome, an array comprising genetic probes covering a whole genome or a substantially complete genome is used. For other types of analyses (i.e., for non genome-wide experiments), the array may contain specific nucleic acid sequences that originate from a gene or chromosomal location, which is known to be associated with a disease or condition, or whose association with a disease or condition is to be tested. Additionally or alternatively, the array may comprise nucleic acid sequences of unknown significance or location. Genetic probes may be used as positive or negative controls (i.e., the nucleic acid sequences may be derived from karyotypically normal genomes or from genomes containing one or more chromosomal abnormalities).

Techniques for the preparation and manipulation of genetic probes are well-known in the art (see, for example, J. Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5^{th} Ed., John Wiley & Sons).

Genetic probes may be obtained and manipulated by cloning into various vehicles. They may be screened and re-cloned or amplified from any source of genomic DNA. Genetic probes may be derived from genomic clones including mammalian and human artificial chromosomes (MACs and HACs, respectively, which can contain inserts from about 5 to 400 kilobases (kb)), satellite artificial chromosomes or satellite DNA-based artificial chromosomes (SATACs), yeast artificial chromosomes (YACs; 0.2-1 Mb in size), bacterial artificial chromosomes (BACs; up to 300 kb); P1 artificial chromosomes (PACs; about 70-100 kb) and the like.

MACs and HACs have been described (see, for example, W. Roush, Science, 1997, 276: 38-39; M. A. Rosenfeld, Nat. Genet. 1997, 15: 333-335; F. Ascenzioni et al., Cancer Lett. 1997, 118: 135-142; Y Kuroiwa et al., Nat. Biotechnol. 2000, 18: 1086-1090; J. E. Meija et al., Am. J. Hum. Genet. 2001, 69: 315-326; and C. Auriche et al., EMBO Rep. 2001, 2: 102-107; see also, for example, U.S. Pat. Nos. 5,288,625; 5,721,118; 6,025,155; and 6,077,697). SATACs can be produced by induced de novo chromosome formation in cells of different mammalian species (see, for example, P. E. Warburton and D. Kiplin, Nature, 1997, 386: 553-555; E. Csonka et al., J. Cell. Sci. 2000, 113: 3207-3216; and G. Hadlaczky, Curr. Opin. Mol. Ther. 2001, 3: 125-132).

Genetic probes may alternatively be derived from YACs, which have been used for many years for the stable propagation of genomic fragments of up to one million base pairs in size (see, for example, J. M. Feingold et al., Proc. Natl. Acad. Sci. USA, 1990, 87:8637-8641; G. Adam et al., Plant J., 1997, 11: 1349-1358; R. M. Tucker and D. T. Burke, Gene, 1997, 199: 25-30; and M. Zeschnigk et al., Nucleic Acids Res., 1999, 27: E30; see also, for example, U.S. Pat. Nos. 5,776,745 and 5,981,175).

BACs may also be used to produce genetic probes for use in the practice of the present invention. BACs, which are based on the E. coli F factor plasmid system, offer the advantage of being easy to manipulate and purify in microgram quantities (see, for example, S. Asakawa et al., Gene, 1997, 191: 69-79; and Y. Cao et al., Genome Res. 1999, 9: 763-774; see also, for example, U.S. Pat. Nos. 5,874,259; 6,183,957; and 6,277,621). PACs are bacteriophage P1-derived vectors (see, for example, P. A. Ioannou et al., Nature Genet., 1994, 6: 84-89; J. Boren et al., Genome Res. 1996, 6: 1123-1130; H. G. Nothwang et al., Genomics, 1997,41: 370-378; L. H. Reid et al., Genomics, 1997, 43: 366-375; and P. Y. Woon et al., Genomics, 1998, 50: 306-316).

Genetic probes may also be obtained and manipulated by cloning into other cloning vehicles such as, for example, recombinant viruses, cosmids, or plasmids (see, for example, U.S. Pat. Nos. 5,266,489; 5,288,641 and 5,501,979).

Alternatively, nucleic acid sequences used as array-immobilized genetic probes may be synthesized in vitro by chemical techniques well-known in the art. These methods have been described (see, for example, S. A. Narang et al., Meth. Enzymol. 1979, 68: 90-98; E. L. Brown et al., Meth. Enzymol. 1979, 68: 109-151; E. S. Belousov et al., Nucleic Acids Res. 1997,25: 3440-3444; M. J. Blommers et al., Biochemistry, 1994, 33: 7886-7896; and K. Frenkel et al., Free Radic. Biol. Med. 1995, 19: 373-380; see also, for example, U.S. Pat. No. 4,458,066).

An alternative to custom arraying of genetic probes is to rely on commercially available arrays and micro-arrays. Such arrays have been developed, for example, by Vysis Corporation (Downers Grove, Ill.), Spectral Genomics Inc. (Houston, Tex.), and Affymetrix Inc. (Santa Clara, Calif.).

The array used by the Applicants in a series of experiments described in Example 3 is the GenoSensor™ Array 300 developed by Vysis. This genomic micro-array enables simultaneously screening for gene amplifications and deletions and provides a sensitivity that allows single gene copy detection. The Vysis arrays consists of 287 probe elements spotted in triplicate and comprises over 1300 gene loci derived primarily from bacterial artificial chromosomes (BACs), including microdeletion regions, important X/Y chromosome targets, aneusomy and aneuploidy of all chromosomes and telomeres.

Hybridization

In the methods of the invention, the CGH array is contacted simultaneously with the test and reference samples under conditions wherein the nucleic acid fragments in the samples can specifically hybridize to the genetic probes immobilized on the array.

The hybridization reaction and washing step(s), if any, may be carried out under any of a variety of experimental conditions. Numerous hybridization and wash protocols have been described and are well-known in the art (see, for example, J. Sambrook et al., “Molecular Cloning: A Laboratory Manual”, 1989, 2^{nd} Ed., Cold Spring Harbour Laboratory Press: New York; P. Tijssen “Hybridization with Nucleic Acid Probes—Laboratory Techniques in Biochemistry and Molecular Biology (Part II)”, Elsevier Science, 1993; and “Nucleic Acid Hybridization”, M. L. M. Anderson (Ed.), 1999, Springer Verlag: New York, N.Y.). The methods of the invention may be carried out by following known hybridization protocols, by using modified or optimized versions of known hybridization protocols or newly developed hybridization protocols as long as these protocols allow specific hybridization to take place.

The term “specific hybridization” refers to a process in which a nucleic acid molecule preferentially binds, duplexes, or hybridizes to a particular nucleic acid sequence under stringent conditions. In the context of the present invention, this term more specifically refers to a process in which a nucleic acid fragment from a test or reference sample preferentially binds (i.e., hybridizes) to a particular genetic probe immobilized on the array and to a lesser extend, or not at all, to other immobilized genetic probes of the array. Stringent hybridization conditions are sequence dependent. The specificity of hybridization increases with the stringency of the hybridization conditions; reducing the stringency of the hybridization conditions results in a higher degree of mismatch being tolerated.

The hybridization and/or wash conditions may be adjusted by varying different factors such as the hybridization reaction time, the time of the washing step(s), the temperature of the hybridization reaction and/or of the washing process, the components of the hybridization and/or wash buffers, the concentrations of these components as well as the pH and ionic strength of the hybridization and/or wash buffers.

In certain embodiments, the hybridization and/or wash steps are carried out under very stringent conditions. In other embodiments, the hybridization and/or wash steps are carried out under moderate to stringent conditions. In still other embodiments, more than one washing steps are performed. For example, in order to reduce background signal, a medium to low stringency wash is followed by a wash carried out under very stringent conditions.

As is well known in the art, the hybridization process may be enhanced by modifying other reaction conditions. For example, the efficiency of hybridization (i.e., time to equilibrium) may be enhanced by using reaction conditions that include temperature fluctuations (i.e., differences in temperature that are higher than a couple of degrees). An oven or other devices capable of generating variations in temperatures may be used in the practice of the methods of the invention to obtain temperature fluctuation conditions during the hybridization process.

It is also known in the art that hybridization efficiency is significantly improved if the reaction takes place in an environment where the humidity is not saturated. Controlling the humidity during the hybridization process provides another means to increase the hybridization sensitivity. Array-based instruments usually include housings allowing control of the humidity during all the different stages of the experiment (i.e., pre-hybridization, hybridization, wash and detection steps).

Additionally or alternatively, a hybridization environment that includes osmotic fluctuation may be used to increase hybridization efficiency. Such an environment where the hyper-/hypo-tonicity of the hybridization reaction mixture varies may be obtained by creating a solute gradient in the hybridization chamber, for example, by placing a hybridization buffer containing a low salt concentration on one side of the chamber and a hybridization buffer containing a higher salt concentration on the other side of the chamber.

In order to create competitive hybridization conditions, the array is contacted simultaneously (i.e., at the same time) with the labeled nucleic acid fragments of the test and reference samples. This may be done by, for example, mixing the test and reference samples to form a hybridization mixture and contacting the array with the mixture.

Highly Repetitive Sequences

In the practice of the methods of the invention, the array is simultaneously contacted with the test and reference samples under conditions wherein the nucleic acid segments in the samples can specifically hybridize to the genetic probes on the array. As mentioned above, the selection of appropriate hybridization conditions will allow specific hybridization to take place. The specificity of hybridization may further be enhanced by inhibiting repetitive sequences.

In certain preferred embodiments, repetitive sequences present in the nucleic acid fragments are removed or their hybridization capacity is disabled. Complex genomes, such as the human genome, comprise different kinds of highly repetitive sequences (e.g., Alu, L1 and satellite sequences), less characterized medium reiteration (MRE) sequences, and simple homo- or oligo-nucleotide tracts. By excluding repetitive sequences from the hybridization reaction or by suppressing their hybridization capacity, one prevents the signal from hybridized nucleic acids to be dominated by the signal originating from these repetitive-type sequences (which are statistically more likely to undergo hybridization). Failure to remove repetitive sequences from the hybridization or to suppress their hybridization capacity results in non-specific hybridization, making it difficult to distinguish the signal from the background noise.

Removing repetitive sequences from a mixture or disabling their hybridization capacity can be accomplished using any of a variety of methods well-known to those skilled in the art. These methods include, but are not limited to, removing repetitive sequences by hybridization to specific nucleic acid sequences immobilized to a solid support (see, for example, O. Brison et al., Mol. Cell. Biol. 1982, 2: 578-587); suppressing the production of repetitive sequences by PCR amplification using adequate PCR primers; inhibiting the hybridization capacity of highly repeated sequences by self-reassociation (see, for example, R. J. Britten et al., Methods of Enzymology, 1974, 29: 363-418); or removing repetitive sequences using hydroxyapatite (which is commercially available, for example, from Bio-Rad Laboratories, Richmond, Va.).

Preferably, the hybridization capacity of highly repeated sequences is competitively inhibited by including, in the hybridization mixture, unlabeled blocking nucleic acids. The unlabeled blocking nucleic acids, which are mixed to the test and reference samples before the contacting step, act as a competitor and prevent the labeled repetitive sequences from binding to the highly repetitive sequences of the genetic probes, thus decreasing hybridization background. In certain preferred embodiments, the unlabeled blocking nucleic acids are Human Cot-1 DNA. Human Cot-1 DNA is commercially available, for example, from Gibco/BRL Life Technologies (Gaithersburg, Md.).

Binding Detection and Data Analysis

The methods of the invention include determining the binding of the individual nucleic acid fragments of the test and reference samples to the individual genetic probes immobilized on the array in order to obtain a relative binding pattern. In array-based CGH, determination of the relative binding is carried out by analyzing the labeled array which results from co-hybridization of the two differentially labeled samples.

In certain embodiments, determination of the relative binding includes: measuring the intensity of the signals produced by the first detectable agent and second detectable agent at each discrete spot on the array; and determining the ratio of the intensities of the signals for each spot. Ratios of the signal intensity from the samples at discrete locations on the array reflect the competitive hybridization of DNA sequences in the test and reference samples. The relative binding pattern determined over the array (i.e., over a substantially complete genome or a subset of a genome) therefore provides a locus-by-locus measure of DNA copy number variations.

Analysis of the labeled array may be carried out using any of a variety of means and methods, whose selection will depend on the nature of the first and second detectable agents.

In preferred embodiments, the first and second detectable agents are fluorescent dyes and the relative binding is detected by fluorescence. To allow determination of the relative hybridization, the first and second fluorescent labels should constitute a matched pair that is compatible with the detection system of the array-based CGH instrument to be used. Matched pairs of fluorescent labeling dyes preferably produce signals that are spectrally distinguishable. For example, the fluorescent dyes in a matched pair do not significantly absorb light in the same spectral range (i.e., they exhibit different absorption maxima wavelengths) and can be excited (for example, sequentially) using two different wavelengths. Alternatively, the fluorescent dyes in a matched pair emit light in different spectral ranges (i.e., they produce a dual-color fluorescence upon excitation).

Pairs of fluorescent labels are known in the art (see, for example, R. P. Haugland, “Molecular Probes: Handbook of Fluorescent Probes and Research Chemicals 1992-1994”, 5^{th} Ed., 1994, Molecular Probes, Inc.). Exemplary pairs of fluorescent dyes include, but are not limited to, rhodamine and fluorescein (see, for example, J. DeRisi et al., Nature Gen. 1996, 14: 458-460); Spectrum Red™ and Spectrum Green™ (commercially available from Vysis, Inc., (Downers Grove, Ill.)); and Cy-3™ and Cy-5™ (commercially available from Amersham Life Sciences (Arlington Heights, Ill.)).

Analysis of a fluorescently labeled CGH array usually comprises: detection of multiple fluorescence over the whole array, image acquisition, quantitation of fluorescence intensity from the imaged array, and data analysis.

Methods for the simultaneous detection of multiple fluorescent labels and the creation of composite fluorescence images are well known in the art and include the use of “array reading” or “scanning” systems, such as charge-coupled devices (i.e., CCDs). Any known device or method, or variation thereof, can be used or adapted to practice the methods of the invention (see, for example, Y. Hiraoka et al., Science, 1987, 238: 3641; R. S. Aikens et al., Meth. Cell Biol. 1989, 29: 291-313; A. Divane et al., Prenat. Diagn. 1994, 14: 1061-1069; S. M. Jalal et al., Mayo Clin. Proc. 1998, 73: 132-137; V. G. Cheung et al., Nature Genet. 1999, 21: 15-19; see also, for example, U.S. Pat. Nos. 5,539,517; 5,790,727; 5,846,708; 5,880,473; 5,922,617; 5,943,129; 6,049,380; 6,054,279; 6,055,325; 6,066,459; 6,140,044; 6,143,495; 6,191,425; 6,252,664; 6,261,776; and 6,294,331).

Commercially available microarrays scanners are typically laser-based scanning systems that can acquire two (or more) differentially fluorescent images sequentially (as, for example, in the systems commercially available from PerkinElmer Life and Analytical Sciences, Inc. (Boston, Mass.)) or simultaneously (as, for example, in the systems commercially available from Virtek Vision Inc. (Ontario, Canada) and Axon Instruments, Inc. (Union City, Calif.)). Arrays can be scanned using several different laser intensities in order to ensure the detection of weak fluorescence signals and the linearity of the signal response at each spot on the array (see below). Fluorochrome-specific optical filters may be used during the acquisition of the fluorescent images. Filter sets are commercially available, for example, from Chroma Technology Corp. (Rockingham, Vt.).

Preferably, a computer-assisted imaging system capable of generating and acquiring multicolor fluorescence images from arrays such as those described above, is used in the practice of the methods of the invention. One or more fluorescent images of the labeled array after hybridization may be acquired and stored.

Preferably, a computer-assisted image analysis system is used to analyze the acquired fluorescent images. Such systems allow for an accurate quantitation of the intensity differences and for an easier interpretation of the results. A software for fluorescence quantitation and fluorescence ratio determination at discrete spots on an array is usually included with the scanner hardware. Softwares and hardwares are commercially available and may be obtained from, for example, Applied Spectral Imaging, Inc. (Carlsbad, Calif.); Chroma Technology Corp. (Brattleboro, Vt.); Leica Microsystems, (Bannockburn, Ill.); and Vysis, Inc. (Downers Grove, Ill.). Other softwares are publicly available (e.g., ScanAlyze (http://rana.lbl.gov); M. B. Eisen et al., Proc. Natl. Acad. Sci. USA, 1998, 95: 14863-14868).

Image analysis using a computer-assisted system includes image capture, interpretation of the imaged array (through pre-processing, spot identification, ratio measurement at each spot on the array), and display of the results of the analysis as copy number ratios as a function of location on the (arrayed) genome (i.e., genomic locus).

As described in Example 3, the system used by the Applicants is the micro-array technology system called GenoSensor™ that was developed by Vysis (see U.S. Pat. Nos. 5,830,645 and 6,159,685, each of which is incorporated herein by reference in its entirety). The GenoSensor™ Reader comprises a fluorescent imaging device with a Xenon-illumination source, an automated six-position filter wheel with three filters, a 1.3 million pixel high-resolution cooled CCD camera, an Apple Macintosh G4 computer with a 17″ monitor. The GenoSensor™ software provide results of the karyotype analysis displayed as shown in Table 1 (Example 3).

Accurate determination of fluorescence intensities requires normalization and determination of the fluorescence ratio baseline (A. Brazma and J. Vilo, FEBS Lett. 2000, 480: 17-24). Data reproducibility may be assessed by using arrays on which genetic probes are spotted in duplicate or triplicate. Similarly, genetic probes containing nucleic acid sequences known not to be involved in copy number changes may be present on CGH arrays and used as internal controls. The specificity of the system may be established by performing parallel experiments in which differentially labeled control genomic DNA is compared against itself. Baseline thresholds may also be determined using global normalization approaches such as those used in expression array experiments (M. K. Kerr et al., J. Comput. Biol. 2000, 7: 819-837). Mathematical normalization may be performed to compensate for general differences in the staining intensities of different fluorescent dyes.

Furthermore, control experiments should preferably be carried out to assess the linearity of the relationship between the fluorescence ratio and copy number variations, as this relationship was reported to deviate from linearity at low copy numbers (A. Kallioniemi et al., Science, 1992, 258: 818-821; J. R. Pollack et al., Nature Genet. 1999, 23: 41-46; S. Solinas-Toldo et al., Genes, Chromosomes & Cancer, 1997, 20: 399-407; and D. Pinkel et al., Nature Genet. 1998, 20: 207-211).

Other Array-Based Hybridization Methods for Amniotic Fluid Fetal DNA Analysis

As mentioned above, the analysis of cell-free fetal DNA by array-based hybridization may be carried out using other array-based techniques than array-based comparative genomic hybridization, as long as fetal genomic information may be obtained.

For example, SNP (i.e., Single Nucleotide Polymorphism) arrays, commercially available from, for example, Affymetrix Inc. (Santa Clara, Calif.) or Orchid Biosciences (Princeton, N.J.), may be useful in karyotyping. Multiple chromosomal rearrangements, for example those resulting in loss of heterozygosity (LOH), may be detected using SNP arrays (R. Mei et al., Genome Res. 2000, 10: 1126-1137). SNP arrays have been used in a variety of applications, such as familial linkage studies that aim to map inherited disease or drug susceptibility as well as for tracking de novo genetic alterations. SNP arrays enable whole-genome survey by simultaneously tracking a large number of genetic variations (i.e., single nucleotide polymorphisms) dispersed throughout the genome. SNP arrays may be particularly useful to detect LOH events that do not lead to DNA copy number changes (S. A. Hagstron and T. P. Dryja, Proc. Natl. Acad. Sci. USA, 1999, 96: 2952-2957). Methods of carrying out DNA analysis using SNP arrays are well known in the art. Arrays are being developed (for example, by Affymetrix) with new SNP content and much broader surveying capabilities. Such arrays will find applications in the practice of the methods of the present invention.

The methods of the invention may also be performed using arrays that allow examination of gene variations (e.g., presence of individual base pair changes or mismatches) in particular genes or gene subsets.

III. Prenatal Diagnosis

Practicing the methods of the present invention includes providing a prenatal diagnosis. In certain embodiments, the prenatal diagnosis is provided based on a relative binding pattern that reflects the relative abundance of nucleic acid sequences in a test and reference samples, thereby revealing the presence of chromosomal abnormalities. In other embodiments, the prenatal diagnosis is provided based on fetal genomic information such as gain and loss of genetic material at multiple genomic loci.

Chromosomal Abnormalities and Associated Diseases and Conditions

Chromosomal aberrations that can be detected and identified by the methods of the present invention include numerical and structural chromosomal abnormalities.

For example, the methods of the invention allow for detection of numerical abnormalities, such as those in which there is an extra set(s) of the normal (or haploid) number of chromosomes (triploidy and tetraploidy), those with a missing individual chromosome (monosomy) and those with an extra individual chromosome (trisomy and double trisomy). The presence of an abnormal number of chromosomes in an otherwise diploid organism is called aneuploidy (see, A. C. Chandley, in: “Human Genetics—Part B: Medical Aspects”, 1982, Alan R. Liss: New York, N.Y.). Approximately half of spontaneous abortions are associated with the presence of an abnormal number of chromosomes in the karyotype of the fetus (M. A. Abruzzo and T. J. Hassold, Environ. Mol. Mutagen. 1995, 25: 38-47), which makes aneuploidy the leading cause of miscarriage. Trisomy is the most frequent type of aneuploidy and occurs in 4% of all clinically recognized pregnancies (T. J. Hassold and P. A. Jacobs, Ann. Rev. Genet. 1984, 18: 69-97). The most common trisomies involve the chromosomes 21 (associated with Down syndrome), 18 (Edward syndrome) and 13 (Patau syndrome) (see, for example, G. E. Moore et al., Eur. J. Hum. Genet. 2000, 8: 223-228). Other aneuploidies are associated with Turner syndrome (presence of a single X chromosome), Klinefelter syndrome (characterized by an XXY karyotype) and XYY disease (characterized by an XYY karyotype).

Hybridization analysis of amniotic fluid fetal DNA according to the methods of the present invention may be used to detect numerical chromosomal abnormalities and therefore to diagnose diseases and conditions associated with aneuploidies including, but not limited to: Down syndrome, Edward syndrome and Patau syndrome, as well as Turner syndrome, Klinefelter syndrome and XYY disease. Comparative genomic hybridization has successfully been applied to detect aneuploidy in spontaneous abortions, which demonstrates the utility of using such a technique prenatally (M. Daniely et al., Hum. Reprod. 1998, 13: 805-809).

Other types of chromosomal abnormalities that can be detected and identified by the methods of the present invention are structural chromosomal aberrations. In contrast to numerical chromosomal abnormalities that correspond to gains or losses of entire chromosomes, structural chromosomal aberrations involve portions of chromosomes. Structural chromosomal aberrations include: deletions (e.g., absence of one or more nucleotides normally present in a gene sequence, absence of an entire gene, or missing portion of a chromosome), additions (e.g., presence of one or more nucleotides normally absent in a gene sequence, presence of extra copies of genes (also called duplications), or presence of an extra portion of a chromosome), rings, breaks, and chromosomal rearrangements, such as translocations and inversions.

The methods of the invention may be used to detect chromosomal abnormalities involving the X chromosome. A large number of these chromosomal abnormalities are known to be associated with a group of diseases and conditions collectively termed X-linked disorders. For example, the methods of the invention may be used to detect mutations in the HEMA gene on the X chromosome (Xq28), which are associated with Hemophilia A, a hereditary blood disorder, primarily affecting males and characterized by a deficiency of the blood clotting protein known as Factor VIII resulting in abnormal bleeding.

The methods of the invention may also be used to detect mutations in the DMD gene on chromosome X (Xp21.2), that cause dystrophinopathies such as Duchenne muscular dystrophy. Duchenne muscular dystrophy, which occurs with an incidence rate of approximately 1 in 3,000 live-born male infants, is characterized by progressive muscle weakness starting as early as 2 years of age.

Mutations in the HPRT1 gene located at position q26-q27.2 on the X chromosome may also be detected by the methods of the invention. This chromosomal abnormality is associated with Lesch-Nyhan syndrome, a rare disease which involves disruption of the metabolism of purines. Lesch-Nyhan syndrome is characterized by neurologic dysfunction, cognitive and behavioral disturbances, and uric acid overproduction.

The methods of the invention may also be used to detect mutations in the IL2RG gene at chromosomal location Xq13.1, that are responsible for half of all severe combined immunodeficiency cases. Severe combined immunodeficiency represents a group of rare, sometimes fatal, congenital disorders characterized by little or no immune response. Certain forms of severe combined immunodeficiency are also associated with a mutation in JAK3 (an important signaling molecule activated by IL2RG), located on chromosome 19; other forms result from chromosomal abnormalities involving the ADA gene on chromosome 20.

The inventive methods may also be used to detect an amplification (presence of more than 200 copies) of a CGG motif at one end of the FMR1 gene (Xq27.3) on the X chromosome, which is associated with Fragile X syndrome, the most common inherited form of mental retardation currently known and whose effects are seen more frequently and with greater severity in males than in females.

Other diseases or conditions are known to be associated with amplifications of nucleotide motifs that can be detected by the methods of the invention. For example, myotonic dystrophy, which is a multisystem disorder that affects skeletal muscle and smooth muscle, as well as the eye, heart, endocrine system, and central nervous system, is associated with over-amplification of a CTG motif (>37 copies) on the DMPK gene on chromosome 19 (19q13.2-q13.3). Another example is spinobulbar muscular atrophy, which is a gradually progressive neuromuscular disorder that affects only males, and is associated with amplification of a CAG repeat (>35 copies) in the androgen receptor (AR) gene located on chromosome 11 (Xq11-q12).

In addition to Fragile X syndrome, a number of other retardation disorders are known to result from chromosomal abnormalities involving the terminal regions (or tips) of chromosomes (i.e., telomeres). A large part of the DNA sequence of telomeres are shared among different chromosomes. However telomeres also comprise a unique (much smaller) sequence region that is specific to each chromosome and is very gene-rich (S. Saccone et al., Proc. Natl. Acad. Sci. USA, 1992, 89: 4913-4917). Chromosome rearrangements involving telomeric regions can have serious clinical consequences. For example, submicroscopic subtelomeric chromosome rearrangements have been found to be a significant cause of mental retardation with or without congenital anomalies (J. Flint et al., Nat. Genet. 1995, 9: 132-140; S. J. L. Knight et al., Lancet, 1999, 354: 1676-1681; B. B. de Vries et al., J. Med. Genet. 2001, 38: 145-150; S. J. L. Knight and J. Flint, J. Med. Genet. 2000, 37: 401-409). Telemore regions have the highest recombination rate and are prone to aberrations resulting from illegitimate pairing and crossover. Since the terminal portions of most chromosomes appear nearly identical by routine karyotyping analysis at the 450- to 500-band level, detection of chromosomal rearrangements in these regions is difficult using standard methodologies. The methods of the invention, which exhibit a much higher resolution than conventional karyotyping methods, may be used to detect such subtelomeric rearrangements (J. A. Veltman et al., Am. J. Hum. Genet. 2002, 70: 1269-1276).

Diseases and conditions associated with telomeric abnormalities include, for example, Cri du Chat syndrome, a disease that may account for up to 1% of individuals with severe mental retardation and which is characterized by deletion of the distal portion of chromosome 5. Another example is Wolf-Hirschhorn syndrome, a disorder that is characterized by typical facial features and microcephaly, and may also be accompanied by skeletal anomalies, congenital heart defects, hearing loss, urinary tract malformations and structural brain abnormalities. Wolf-Hirschhorn syndrome is associated with deletion of the distal portion of the short arm of chromosome 4 involving band 4p16. In certain cases, this deletion occurs along with other chromosomal abnormalities such as a ring or unbalanced translocation involving chromosome 4. The methods of the invention may also find applications in basic and clinical research investigations aimed at acquiring a better understanding of the role of subtelomeric rearrangements in a number of conditions associated with mental retardation.

The methods of the invention may also be used to detect chromosomal abnormalities associated with microdeletion/microduplication syndromes. Microdeletion/microduplication syndromes are a collection of genetic syndromes that are associated with small, cryptic or subtle chromosomal structural aberrations (S. K. Shapira, Curr. Opin. Pediatr. 1998, 10: 622-627), a large number of which are beyond the resolution of detection of standard cytogenetic methods. Some microdeletion syndromes are caused by loss of a single gene; others involve multiple genes or an unknown number of genes. Others still are considered contiguous gene deletion syndromes where deletion of physically contiguous genes leads to complex phenotypic abnormalities. Diagnosis of microdeletion/microduplication syndromes is, currently, incomplete without both karyotype analysis and specific FISH assays, therefore these diseases are most frequently not diagnosed prenatally. Furthermore, even when a FISH analysis is ordered, the technique requires at least some knowledge regarding the types and locations of chromosomal aberration(s) expected in order to select useful DNA probes. The CGH methods of the invention, which allow for a genome-wide screening with single gene copy detection, present the advantage that all cell-free fetal DNA analyzed on the micro-array is automatically interrogated for the presence or absence of such chromosomal microdeletions and microduplications.

For example, the methods of the invention may be used to detect deletion of segment q11-q 13 on chromosome 15, which, when it takes place on the paternally derived chromosome 15, is associated with Prader-Willi syndrome (a disorder characterized by mental retardation, decreased muscle tone, short stature and obesity) and which, when it happens on the maternally derived chromosome 15, is linked to Angelman syndrome (a neurogenetic disorder characterized by mental retardation, speech impairment, abnormal gait, seizures and inappropriate happy demeanor).

The methods of the invention may also be used to detect microdeletions in chromosome 22, for example those occurring in band 22q11.2, which are linked to DiGeorge syndrome, an autosomal dominant condition that is found in association with approximately 10% of cases in prenatally-ascertained congenital heart disease.

The methods of the invention may also be used to diagnose Smith-Magenis syndrome, the most frequently observed microdeletion syndrome. Smith-Magenis syndrome is characterized by mental retardation, neuro-behavorial anomalies, sleep disturbances, short stature, minor cranofacial and skeletal anomalies, congenital heart defects and renal anomalies. It is associated with an interstitial deletion of the chromosome band 17p11.2.

The methods of the invention may also be used to detect a microdeletion involving the CREBBP gene on chromosome 16 (16p13.3), which is associated with Rubinstein-Taybi syndrome, a disorder characterized by moderate-to-severe mental retardation, distinctive facial features and short stature.

The methods of the invention may also be used to detect micro-rearrangements within the LIS1 gene in chromosome band 17p13.3, which are associated with Miller-Dieker syndrome, a multiple malformation disorder characterized by classical lissencephaly (i.e., smooth brain), a characteristic facial appearance and sometimes other birth defects. Miller-Dieker syndrome is considered a contiguous gene deletion syndrome. In Miller-Dieker patients, a deletion of the LIS1 gene is always accompanied with telemoric loci in excess of 250 kb.

The methods of the invention may also be used to detect a deletion at location q11.23 on chromosome 7, which is associated with Williams syndrome, a developmental disorder that includes cardiovascular abnormalities, dysmorphic facial features, developmental delay with a unique cognitive profile, infantile hypercalcaemia and growth retardation.

The methods of the invention are particularly useful when a disease or condition is associated with multiple different chromosomal abnormalities. For example, Charcot-Marie-Tooth (CMT) hereditary neuropathy refers to a group of disorders characterized by a chronic motor and sensory polyneuropathy and associated with chromosomal abnormalities involving the PMPP2 gene on chromosome 17 (17p11.2), the MPZ gene on chromosome 1 (1q22), the NEFL gene on chromosome 8 (8q21), the GJB1 gene on chromosome X (Xq13.1), the EGR2 gene on chromosome 10 (10q21.1-q22.1), and the PRX gene on chromosome 19 (19q13.1-q13.2).

Other chromosomal abnormalities that can be detected and identified by the methods of the invention include, for example, a segmental duplication of a subregion on chromosome 21 (such as 21q22), which can be present on chromosome 21 or another chromosome (i.e., after translocation) and is associated with Down syndrome.

Mutations in the CFTR gene on chromosome 7 (7q31.2) can also be detected by the methods of the invention. Certain mutations in the CFTR gene are associated with cystic fibrosis, the most common fatal genetic disease in the US today. Cystic fibrosis is characterized by impaired breathing due to copious, viscous mucus clogging respiratory passages, poor digestion reflecting pancreatic and intestinal insufficiency, and a salty sweat. About 70% of mutations observed in cystic fibrosis patients result from deletion of three base pairs in CFTR's nucleotide sequence.

The methods of the invention may also be used to detect a deletion of a gene called Rb on chromosome 13 (13q14), which is associated with the hereditary form of retinoblastoma. Retinoblastoma occurs in early childhood and leads to the formation of tumors in both eyes. Left untreated, retinoblastoma is most often fatal. However, a survival rate over 90% is achieved with early post-natal diagnosis and modern methods of treatment.

The methods of the invention may also be used to detect a point mutation in the HBB gene found on chromosome 11 (11p15), which is associated with sickle cell anemia, the most common inherited blood disease in the US. Symptoms of sickle cell anemia include chronic hemolytic anemia and severe infections, as well as episodes of pain.

The methods of the invention may also be used to detect deletions involving chromosomal region 11p13, which are known to be associated with different syndromes such as Wilms tumor (a cancer of the kidneys affecting children), aniridia (a disease of the eyes), genitourinary malformation, and mental retardation.

The methods of the invention may also be used to detect chromosomal abnormalities affecting the GAB gene on chromosome 1 (1q21), which are known to be associated with Gaucher disease, an inherited illness which encompasses a continuum of clinical findings from a prenatal-lethal form to an asymptomatic form.

The methods of the invention may also be used to detect chromosomal abnormalities involving the FBN1 gene on chromosome 15 (15q21.1), which is associated with Marfan syndrome, a systemic disorder of connective tissue with a high degree of variability in the clinical manifestations, which involve the ocular, skeletal and cardiovascular systems.

Prenatal Diagnosis

In certain embodiments, the methods of the invention are performed when the pregnant woman is 35 or older. The most common factor associated with high risk outcome of pregnancy is advanced maternal age. In women over the age of 35, the risk of chromosomal abnormality (1% or higher) presumably exceeds the risk of amniocentesis, which explains that more than 90% of amniocenteses are performed on women of advanced maternal age. Yet it has been estimated that up to 80% of Down syndrome infants are born to women under age 35 (L. B. Holmes, New Eng. J. Med. 1978, 298: 1419-1421), who are generally not considered candidates for amniocentesis. This situation has persuaded some investigators to suggest extending the availability of amniocentesis to all women who ask for such a prenatal test.

In other embodiments, the methods of the invention are performed when the fetus carried by the pregnant woman is suspected of having a chromosomal abnormality or when the fetus is suspected of having a disease or condition associated with a chromosomal abnormality. For example, such situations may arise when a previous child of the couple of prospective parents has a chromosomal abnormality, when there is a case of parental chromosomal rearrangement, when there is a case of family history of late-onset disorders with genetic components, when a maternal serum screening test comes back positive, documenting, for example, an increased risk of fetal neural tube defects and/or fetal chromosomal abnormality, or in case of an abnormal fetal ultrasound examination, for example, one that revealed signs known to be associated with aneuploidy.

IV. Methods of Testing Amniotic Fluid Fetal DNA

In another aspect, the present invention provides methods of using array-based comparative genomic hybridization analysis of amniotic fluid fetal DNA as a research tool. The inventive methods may be used to compare the selectivity and specificity of detection of small or subtle chromosomal rearrangements (i.e., micro-abnormalities) by array-based CGH and by other molecular cytogenetic methods such as FISH. The inventive methods may also be used to detect and identify chromosomal micro-abnormalities that are beyond the limits of detection of standard metaphase chromosome analysis techniques such as metaphase CGH.

Selectivity and Specificity of Detection of Chromosomal Micro-Abnormalities by Array-Based CGH

In the methods of testing of the present invention, a test sample of amniotic fluid fetal DNA known to contain a chromosomal micro-abnormality is tested against a reference sample of control genomic DNA with a normal (euploid) karyotype. Chromosomal micro-abnormalities are defined as small, cryptic or subtle chromosomal abnormalities that are not detectable or are difficult to detect with accuracy using standard metaphase chromosome analysis techniques. Chromosomal micro-abnormalities include microadditions, microdeletions, microduplications, microinversions, microtranslocations, subtelomeric rearrangements and any combinations thereof.

The practice of the inventive methods includes determining the karyotype of the test sample of amniotic fluid fetal DNA by FISH. FISH (or fluorescence in situ hybridization) is a molecular cytogenetic technique in which fluorescent gene probes are used to determine the presence or absence of chromosomes, DNA specific sequences or genes. FISH can be used to elucidate subtle chromosomal rearrangements which cannot be detected by conventional banding techniques. However, such screening requires prior knowledge as to the suspected chromosomal abnormality(ies).

The karyotype (or presence and identification of a particular micro-abnormality) of the test sample determined by FISH analysis is then compared to the results obtained by array-based comparative genomic hybridization. This comparison may include evaluation of the degree of consistency between the two karyotyping methods (i.e., FISH and array-based CGH), comparison of the sensitivity and/or selectivity of detection by both methods of the particular chromosomal micro-abnormality present in the genome of the test sample.

The degree of consistency, sensitivity of detection and selectivity of detection by array-based comparative genomic hybridization and by FISH may be catalogued as a function of chromosomal micro-abnormality present in the genome of the test sample.

Detection and Identification of Chromosomal Micro-Abnormalities

The present invention also provides methods for detecting and identifying chromosomal abnormalities that are beyond the limits of detection of conventional metaphase chromosome analysis techniques. In particular, the present invention provides methods for detecting and identifying, by array-based CGH analysis of amniotic fluid fetal DNA, chromosomal micro-abnormalities that are not detected by metaphase CGH analysis with a standard 550 band level of resolution.

The inventive methods require developing case-control sets of matched test and reference samples. Test samples of amniotic fluid fetal DNA to be used in the practice of the methods of the invention originate from fetuses determined to have multiple congenital anomalies by sonographic examination and whose genome have been shown to be karyotypically normal by metaphase CGH. Reference samples of control amniotic fluid fetal DNA originate from fetuses with a normal sonographic examination and a normal karyotype. Preferably, the samples are matched for fetal gender, site of sample acquisition, gestational age, and storage time.

Ultrasonography is a non-invasive procedure in which high frequency sound waves are used to produce visible images from the pattern of echos made by different tissues and organs. In prenatal diagnosis, ultrasonography examination is used to determine the size and position of the fetus, the size and position of the placenta, the amount of amniotic fluid, and the appearance of fetal anatomy. Ultrasound examinations can reveal the presence of congenital anomalies (i.e., functional, anatomical or structural malformations involving different organs including the brain, heart, lungs, kidneys, liver, bones, and intestinal tract). An abnormal ultrasound is one of the most common indications for amniocentesis as chromosomal defects are known to be associated with certain sonographic features, such as biometric parameters (e.g., short length of femur and humerus, pyelextasis, large nuchal fold, ventriculomegaly, early fetal growth restriction) and morphological signs (e.g., choroids plexys cysts, echogenic bowel, echogenic intracardiac focus).

Analysis by array-based comparative genomic hybridization of amniotic fluid fetal DNA originating from a fetus with multiple congenital anomalies will allow detection and identification of chromosomal abnormalities that are not detected by metaphase CGH, which will demonstrate that the inventive methods add significant clinical information to that which is currently provided by standard metaphase karyotype.

Array-based hybridization analysis of amniotic fluid fetal DNA (in particular array-based comparative genomic hybridization analysis) is therefore expected to have broad applications in the area of prenatal diagnostics. The present inventive methods, which do not require any lengthy enrichment steps, thereby significantly reducing the test time and labor, allow for the rapid identification of genetic abnormalities as compared to conventional methodologies such as metaphase chromosome analysis. Furthermore, array-based CGH is a multiplex technology that permits the simultaneous detection of copy number changes across the entire genome starting with limiting amounts of amniotic fluid. No prior knowledge of genomic information in the areas where chromosomal abnormalities may have occurred is required for array-based CGH analyses, and any chromosomal/genomic region can potentially be tested without prior studies or tests. In addition, the present invention provides higher resolution for the detection and identification of chromosomal abnormalities in amniotic fluid fetal DNA than standard metaphase chromosome analysis. This may be used in the prenatal diagnosis of microdeletion microduplication syndromes that are often not easily diagnosed prenatally as well as in the detection of subtelomeric rearrangements that are known to be a significant cause of genetic disorders. The methods of the invention thus permit karyotypic analyses to be conducted more widely, more rapidly and more accurately than was previously feasible.

V—Kits

In another aspect, the present invention provides kits comprising materials useful for carrying out the methods of the invention.

Inventive kits contain the following components: materials to extract cell-free fetal DNA from a sample of amniotic fluid; an array comprising a plurality of genetic probes, wherein each genetic probe is immobilized to a discrete spot on a substrate surface to form the array and wherein together the genetic probes comprise a substantially complete genome or a subset of a genome; and instructions for using the array according to the methods of the invention.

The inventive kits may, additionally, contain materials to label a first sample of DNA with a first detectable agent and a second sample of DNA with a second detectable agent. Preferably, when the inventive kits comprise materials to label samples with detectable agents, the first detectable agent comprises a first fluorescent label and the second detectable agent comprises a second fluorescent label. Preferably, the first and second fluorescent labels produce a dual-color fluorescence upon excitation. For example, an inventive kit may contain materials to differentially label two samples of DNA with Cy-3™ and Cy-5™, or with Spectrum Red™ and Spectrum Green™.

The inventive kits may, additionally, contain a reference sample of control genomic DNA, wherein the reference sample comprises a plurality of nucleic acid segments comprising a substantially complete genome with a known karyotype. In certain embodiments, the genome of the reference sample is karyotypically normal. In other embodiments, the genome of the reference sample is karyotypically abnormal (for example, it is known to contain a chromosomal abnormality such as an extra individual chromosome, a missing individual chromosome, an extra portion of a chromosome, a missing portion of a chromosome, a ring, a break, a translocation, an inversion, a duplication, a deletion, or an addition). The inventive kits may, for example, contain two reference samples of control genomic DNA: one sample with a normal, female karyotype and another sample with a normal, male karyotype. Alternatively, the inventive kits may contain three reference samples of control genomic DNA: a first sample with a normal, female karyotype, a second sample with a normal, male karyotype and a third sample with a karyotypically abnormal karyotype.

In certain embodiments, the inventive kits, additionally, contain hybridization and wash buffers.

In other embodiments, the inventive kits, additionally, contain unlabeled blocking nucleic acids such as Human Cot-1 DNA.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Most of the experimental results presented below have been described by the Applicants in a recent scientific publication (P. B. Larrabee et al., Am. J. Hum. Genet., 2004, 75: 485-491), which is incorporated herein by reference in its entirety.

### Example 1

### Amniotic Fluid Fetal DNA Isolation and Preliminary Tests

Frozen amniotic fluid supernatant specimens (38) were obtained from the Tufts-New England Medical Center (Tufts-NEMC) Cytogenetics Laboratory (D. W. Bianchi et al., Clin. Chem. 2001, 47: 1867-1869). All samples were collected for routine indications, such as advanced maternal age, abnormal maternal serum screening results, or detection of a fetal sonographic abnormality. The standard protocol in the Cytogenetics Laboratory is to centrifuge the amniotic fluid sample upon receipt, place the cell pellet into tissue culture, assay an aliquot of the fluid for alpha-fetoprotein and acetyl cholinesterase levels, and store the remainder at −20° C. as a back-up in case of assay failure. After six months, the frozen amniotic fluid supernatant samples are normally discarded.

The frozen fluid samples obtained from the Cytogenetics Laboratory were initially thawed at 37° C. and then mixed with a vortex for 15 seconds. An aliquot of 500 μL of fluid was spun at 14,000 rpm in a microcentrifuge to remove any remaining cells. A final volume of 400 μL of the supernatant was used for extraction of DNA using the “Blood and Body Fluid” protocol as described by Qiagen.

Real-time quantitative PCR analysis was performed using a Perkin-Elmer Applied Biosystems (PE-ABI) 7700 Sequence Detector. Analysis was based on the 5′-to-3′ exonuclease activity of the Tap DNA polymerase, using the FCY locus as a basis for detecting male DNA if the fetus was male. The FCY primers were derived from the Y-chromosome-specific sequence Y49a (DYSI) (G. Lucotte et al., Mol. Cell. Probes, 1991, 5: 359-363). The FCY amplification system consisted of the amplification primers FCY-F (5′-TCCTGCTTATCCAAATTCACCAT-3′) and a dual-labeled fluorescent TaqMan probe, FCY-T:

(5′-FAMAAGTCGCCACTGGATATCAGTTCCCTTCTTAMRA-3′). The β-globin gene was used to confirm the presence of DNA and estimate its overall concentration.

Amplification reactions were set up as described previously by Y. M. D. Lo et al. (Am. J. Hum. Genet. 1998, 62: 768-775), except that each primer was used at 100 nM and the probe was used at 50 nM. Amplification data were collected by the 7700 Sequence Detector and analyzed using the Sequence Detection System software, Ver. 1.6.3 (PE-ABI). Each sample was run in quadruplicate with the mean results of the four reactions used for further calculations. An amplification calibration curve was created using titrated purified male DNA. The extractions and subsequent quantitative assays were performed twice for each sample, with the mean of the two results used for final analysis.

In 21 samples, the known fetal karyotype was 46, XX (normal female), in 15 samples the known fetal karyotype was 46, XY (normal male), and in two samples, the known karyotype was 47, XY, +21 (male fetus with Down syndrome). However, the samples were coded and analyzed blindly. The mean amount of β-globin DNA detected was 3,427 GE/mL (range 293-15,786). There was no correlation between gestational age and the total amount of DNA detected. In the female fetuses 0 GE/mL of DYSI DNA was detected in the amniotic fluid. The mean value of DYSI DNA detected in male fetuses was 2,668 GE/mL (range 228-12,663 GE/mL). Linear regression analysis showed a correlation between fetal DNA and gestational age (r=0.6225, p=0.0231). In all 38 cases, the predicted fetal gender was correct. The results were statistically significant p<0.0001, by Fisher's exact test). In the cases of fetal Down syndrome, there was no elevation of the amount of fetal DNA compared to the samples obtained from fetuses with a normal male karyotype.

These data show that there is 100-200 fold more fetal DNA per milliliter of fluid in the amniotic fluid compartment, as compared with maternal serum and plasma. Therefore, amniotic fluid appears as a previously unappreciated rich source of fetal nucleic acids that can be obtained relatively easily by using standard procedures.

### Example 2

### Molecular Karyotyping using Cell-Free Fetal DNA from Amniotic Fluid

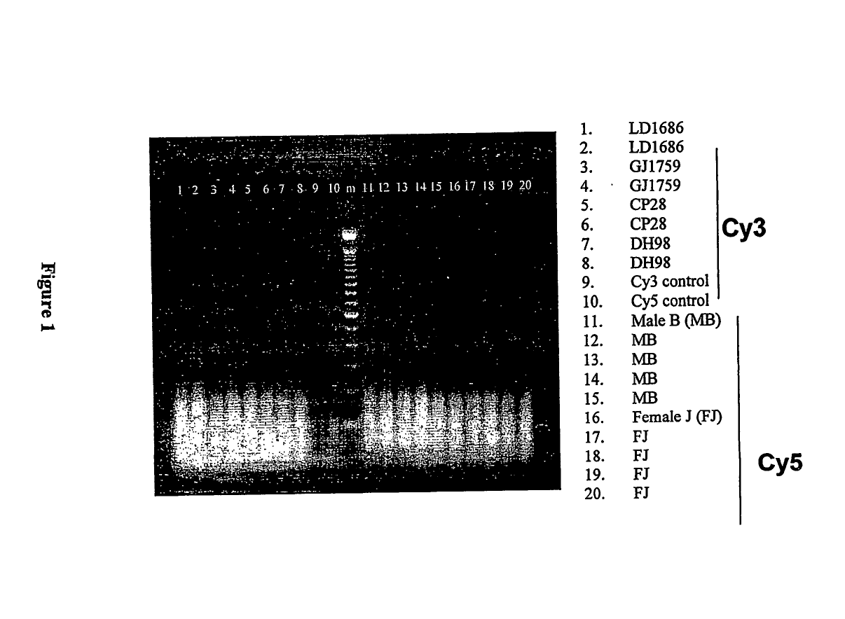

To determine if cell-free fetal DNA in amniotic fluid could be used for molecular karyotyping, cell-free DNA was extracted from eight frozen amniotic fluid supernatant samples from four known euploid males and four known euploid females. Each sample was ≧10 mL in volume and yielded between 200 and 900 ng of DNA. The samples were sent to Vysis for analysis. The results obtained by Vysis confirmed the quantity of DNA present. The concentration of DNA was adjusted to 25 ng/μL. Samples were labeled with Cy-3™ and Cy-5™ according to the current labeling protocol for the GenoSensor™ Array 300. For each sample, reference male and female DNA of equal quantity was labeled for CGH. After DNase digestion, samples were visualized on a 2% agarose/ethidium bromide gel. As shown in FIG. 1, DNA from samples and controls demonstrated uniform amplification and labeling.

Samples were combined, added to hybridization buffer, pre-incubated, and hybridized to the CGH arrays for 72 hours at 37° C. The initial set of four samples (two male, two female) failed to produce conclusive data due to internal reference problems. However, the second set of samples did provide significant data, allowing the co-investigators (who were blinded) to correctly identify the fetal gender in all four cases. The results obtained for the second set of samples are presented in Table 1.

When the test DNA was from a male fetus, Y chromosome genomic sequences (SRY and AZFa) were significantly elevated compared with the reference female DNA (expected ratio >1, observed ratios between 1.37 and 2.18, p<0.01). Similarly, when the test DNA was male, X chromosome sequence (STS3′, STS5′, KAL, dystrophin exons 45-51, and AR3′) signals were significantly decreased compared to the reference female DNA (expected ratio 0.5, observed ratios between 0.46 and 0.71, p<0.01). When the test DNA derived from a female fetus, the Y chromosome sequences were significantly decreased compared to reference male DNA (expected ratio <1, observed ratios between 0.43 and 0.65), and X chromosome sequences were significantly increased when compared to male reference DNA (expected ratio ˜2, observed ratios between 1.30 and 1.86, p<0.01).

The results of these experiments allow to conclude that the gender of the fetuses GJ1759 and LD1686 is male, while samples CP28 and DH98 are female.

TABLE 1
Loci detected as changes with a p value of <0.01 for amniotic DNA samples

| | | | Mean Bias Corrected T/R | | | | | | | |
|---|---|---|---|---|---|---|---|---|---|---|
| | | | GJ1759/ | GJ1759/ | LD1686/ | LD1686/ | CP28/ | CP28/ | DH98/ | DH98/ |
| Clone name | Cyto Location | # Spots | male B | female J | male B | Female J | male B | female J | male B | female J |
| INS | 11p tel | 3 | . | . | 1.4450 | 1.4457 | | . | . | 1.2187 |
| CDKN1C(p57) | 11p15.5 | 3 | . | 1.2433 | . | . | . | . | . | . |
| FES | 15q26.1 | 3 | . | . | 1.3587 | 1.4493 | 1.2910 | . | . | . |
| 282M15/SP6 | 17p tel | 3 | . | . | . | . | . | . | . | . |
| TK1 | 17q23.2-q25.3 | 3 | . | . | . | . | . | 1.2333 | . | . |
| 1PTEL06 | 1p tel | 3 | 1.2363 | . | 1.4687 | 1.5227 | 1.3380 | . | . | 1.2657 |
| CEB108/T7 | 1p tel | 3 | . | . | 1.3743 | . | . | . | . | . |
| TNFRSF6B(DCR3) | 20q13 | 3 | . | . | . | 1.3680 | . | . | . | . |
| BCR | 22q11.23 | 3 | . | . | . | . | . | 1.2723 | . | . |
| p44S10 | 3p14.1 | 3 | 0.6953 | . | . | . | . | . | . | . |
| RASSF1 | 3p21.3 | 3 | . | . | . | . | . | . | 1.3040 | . |
| DHFR, MSH3 | 5q11.2-q13.2 | 3 | . | . | . | . | . | . | . | 1.2027 |
| D6S434 | 6q16.3 | 3 | . | . | . | 0.7040 | . | . | . | . |
| DXS580 | Xp11.2 | 3 | . | 0.7857 | . | 0.7053 | . | . | . | . |
| DMD exon 45-51 | Xp21.1 | 3 | . | 0.5933 | . | 0.4793 | 1.3680 | . | 1.4377 | . |
| KAL | Xp22.3 | 3 | . | 0.7083 | . | 0.6527 | 1.4633 | . | 1.3637 | . |
| STS 3′ | Xp22.3 | 3 | . | 0.5887 | . | 0.6037 | 1.6970 | . | 1.4893 | . |
| STS 5′ | Xp22.3 | 3 | . | 0.6770 | . | 0.6017 | 1.4180 | . | 1.3413 | . |
| AR 3′ | Xq11-q12 | 3 | . | 0.6373 | . | 0.5823 | 1.5153 | . | 1.3747 | . |
| DXS7132 | Xq12 | 3 | . | 0.8203 | . | 0.6787 | . | . | . | . |
| XIST | Xq13.2 | 3 | . | 0.7363 | . | 0.6890 | . | . | . | . |
| OCRL1 | Xq25 | 3 | . | 0.6163 | . | 0.5877 | 1.7900 | . | 1.6000 | . |
| SRY | Yp11.3 | 3 | . | 2.0323 | . | 2.1090 | 0.4810 | . | 0.6627 | . |
| AZFa region | Yq11 | 3 | . | 1.2900 | . | * | 0.6557 | . | 0.7690 | . |

* T/R ratio for AZFa region in LD1686/Female J AZFa hyb was 1.2 but the Pvalue did not show due to higher CVs on these spots.

The preliminary data show that cell-free fetal DNA found in amniotic fluid is of sufficient quality and quantity to be labeled and used on a CGH array for molecular karyotyping to determine copy number. The amniotic fluid DNA labels and hybridizes well to genomic microarrays. This implies that there is sufficient DNA present in the amniotic fluid that is of good quality (i.e., not degraded) so that it should be possible to test the hypothesis that cell-free fetal DNA in amniotic fluid can provide more clinical information than that obtained by the current metaphase karyotype. For example, cell-free DNA from amniotic fluid can provide copy number of genes and the deletion of genes that can not be detected at the current microscopic level of visualization.

### Example 3

### Use of Amniotic Fluid Cell-Free Fetal DNA in CGH Microarrays to Generate a Molecular Karyotype: Preliminary Studies

In a typical analysis, fetal DNA is extracted from stored amniotic fluid supernatant samples with normal and abnormal karyotypes. The samples are then sent to Vysis for analysis. The samples are hybridized to euploid male and euploid female reference DNA on CGH microarrays. The hybridization data is then analyzed and interpreted by the Applicants at Tufts/New England Medical Center.

Vysis has developed a novel microarray technology system that permits simultaneous assessment of multiple genomic targets. The GenoSensor™ system consists of the following hardware: MacIntosh G3 PowerPC computer with 17″ high resolution display monitor, 1.3 million pixel high-resolution cooled CCD camera, custom-designed optics, automated 6-position filter wheel with 3 filters, and xenon illumination source. The microarray consists of over 1,300 gene loci derived primarily from bacterial artificial chromosomes (BACs) as well as test and reference DNA that have been labeled with fluorophores. Using CGH, multiple clones of gene targets can be measured by analysis of fluorescent color ratios of the individual gene targets. The GenoSensor™ reader uses high resolution imaging technology to automatically acquire fluorescent images of the microarray within one minute. The reader software interprets the array image and determines copy number changes between the test and reference DNA.

Under an IRB-approved protocol, greater than 1300 amniotic fluid supernatant specimens have been collected and stored (at −20° C.). Twenty three (23) case-control sets consisting of amniotic fluid from a fetus with a known aneuploidy (such as trisomies 13, 18, 21, or XXY), and at least five control specimens from euploid fetuses matched for fetal gender, site of sample acquisition, gestational age, and time in freezer storage have been developed. In addition, multiple samples from fetuses with chromosomal deletions or rearrangements are also available.

In a series of preliminary experiments, twelve frozen samples of amniotic fluid (from six fetuses with aneuploid karyotypes and six fetuses with normal karyotypes) were used and amniotic fluid fetal DNA extracted from these samples was studied on Vysis' microarray. The goal of these experiments was to identify whole chromosomes changes, including aneuploidy and gender.

In these experiments, all residual cells were removed from the amniotic fluid samples before DNA extraction. One hundred ng of each DNA sample was used per array. Test and reference samples were labeled with Cy-3™ and Cy-5™, respectively and hybridized as described previously. Although hybridization was initially poor for all samples, adjusting the pH of the DNA samples to seven was found to increase the hybridization sensitivity and specificity. Two samples analyzed under these conditions were correctly identified as male, as the majority of X chromosome markers had significantly decreased hybridization compared to the reference female DNA and the SRY locus had significantly increased hybridization compared to the female reference, after normalization of the data. One of the two samples had been determined to originate from a fetus with trisomy 21 (karyotype 47, XY, +21, sample 02-1636). Analyzed by array-based comparative genomic hybridization, this sample was found to exhibit an increased hybridization on five of six chromosome 21 markers compared to the euploid reference DNA. However, the p-values were lower than 0.05 for only four of these markers and none of the p-values were lower than 0.005, which is the rigorous cutoff used by Vysis for these analyses.

These preliminary experiments allowed gender identification with 100% accuracy and led to encouraging conclusions regarding the ability of microarrays to detect aneuploidy.

In a second series of experiments, nine frozen amniotic fluid samples with known euploid karyotypes were used, and DNA was extracted from the cell-free supernatant fraction, as previously described. In order to maximize the amount of fetal DNA available for analysis, a second centrifuge spin was not performed to remove possible residual cells after thawing and prior to extraction. DNA was also extracted separately from samples of cultured amniocytes corresponding to eight of these samples. These amniocytes had been harvested and frozen after the cytogenic karyotype was obtained. All DNA samples were eluted into TE buffer with a neutral pH of seven.

DNA quantification was carried out by real-time PCR method and using the Hoechst fluorometry method. One sample (PR 861) was selected as a pilot sample, to determine if hybridization would work well. The amniotic fluid cell-free DNA, DNA from amniocytes, and male and female reference DNA samples were all labeled separately, as described above. The amniotic fluid cell-free DNA was hybridized to two microarrays: one with a female reference DNA and one with a male reference DNA. The DNA from amniocytes was also similarly hybridized to two microarrays. Both the amniotic fluid cell-free DNA and the DNA from amniocytes were found to hybridize well to the microarrays, and the results had few false positives and negatives. This sample was correctly identified as female.

Next, the remaining eight amniotic fluid cell-free DNA samples and seven DNA samples from amniocytes were hybridized to microarrays using female reference DNA. All samples hybridized well except for one amniotic fluid DNA sample (JH769), which was not informative. The remaining samples had few false positives or negatives. Clone-clone variability was slightly higher in amniotic fluid cell-free DNA samples compared to DNA samples extracted from intact, cultured amniocytes, suggesting that the DNA quality might be lower in the cell-free samples.

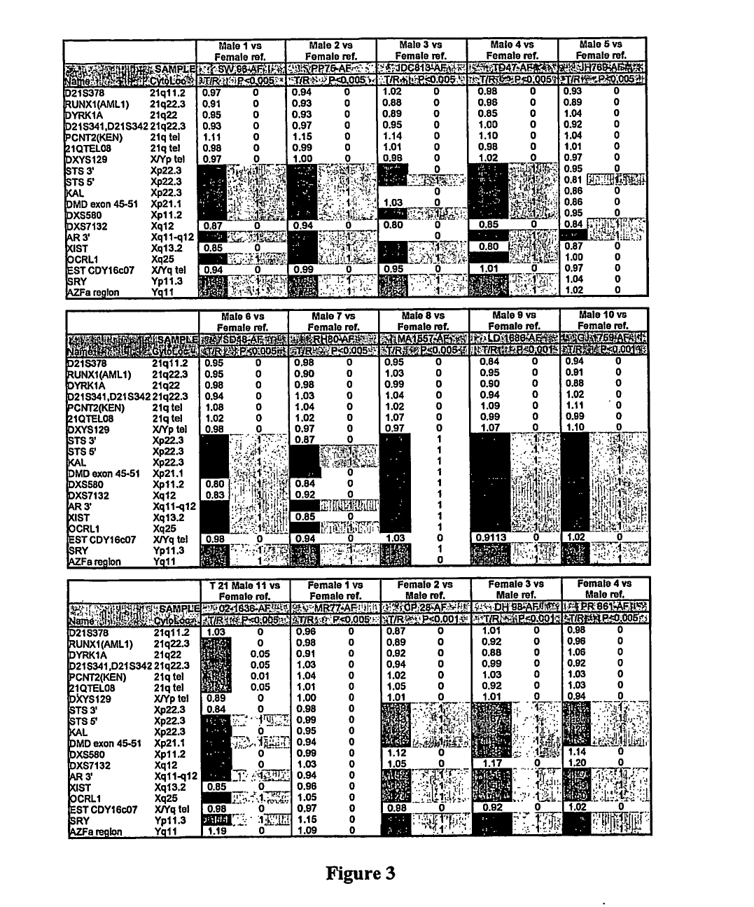

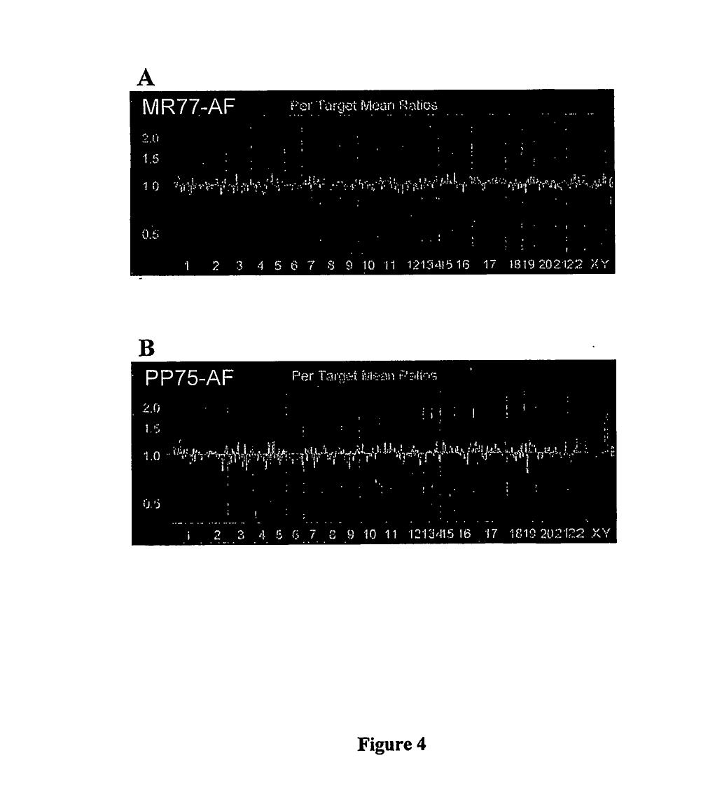

Eight of the nine amniotic fluid cell-free DNA samples and all eight DNA samples from amniocytes led to correct identification of gender when hybridized to the Vysis GenoSensor™ microarray. One amniotic fluid cell-free sample (JH769) was not informative. Results obtained in both series of preliminary experiments are reported in the table of FIG. 3 and in FIG. 4. Overall, the data obtained shows that cell-free fetal DNA extracted from amniotic fluid supernatant can be a reliable source of nucleic acids for molecular karyotyping using microarrays.

### Example 4

### Use of Amniotic Fluid Cell-Free Fetal DNA in CGH Microarrays to Generate a Molecular Karyotype: Complete Study

In a more complete study, a total of 28 cell-free fetal DNA samples (19 euploid and 9 aneuploid) and the 8 corresponding euploid amniocyte DNA samples were considered.

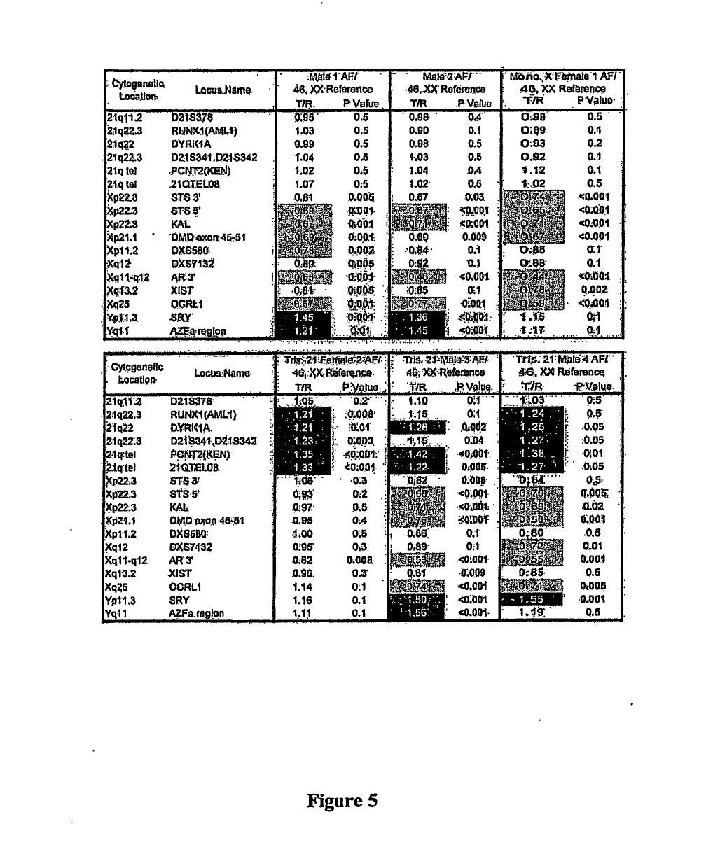

Data are presented for the informative 17 of 28 microarrays hybridized with cell-free fetal DNA extracted from amniotic fluid and for 7 of 8 microarrays hybridized with DNA extracted from residual cultured amniocytes. The karyotypes for the 17 cell-free fetal DNA samples were 46,XX (4 out of 17), 46, XY (9), 47,XY,+21 (2), 47,XX,+21 (1), and 45,X (1). Of the 17 samples in this group, 7 had corresponding cellular samples. FIGS. 5, 6 and 7 show data from all 17 cell-free fetal DNA samples, representing chromosomes X, Y, and 21 for each of these microarrays. As reported above, gender identification was 100% accurate.

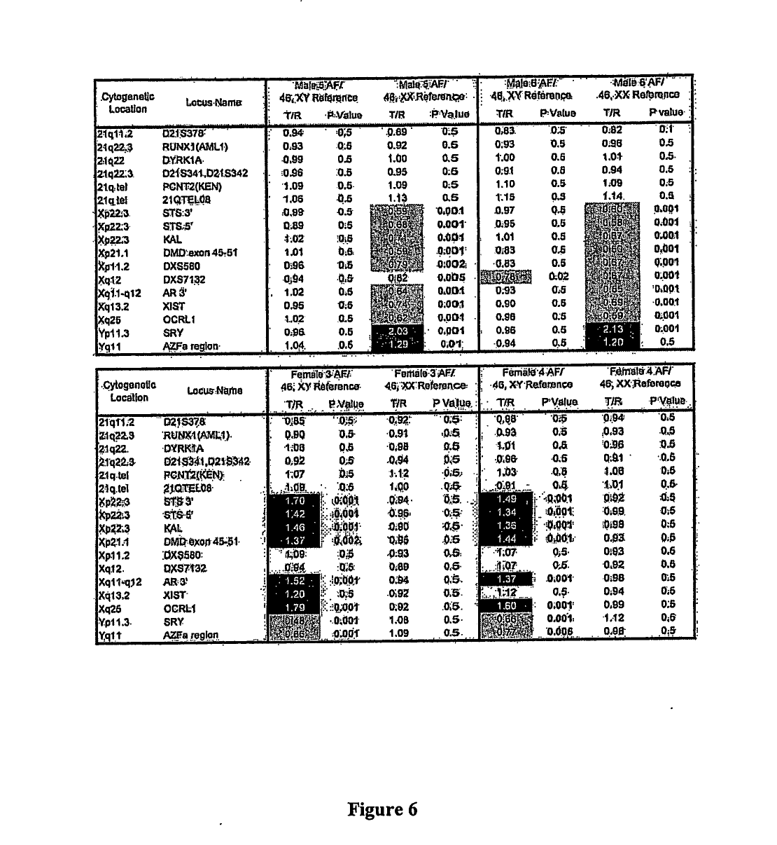

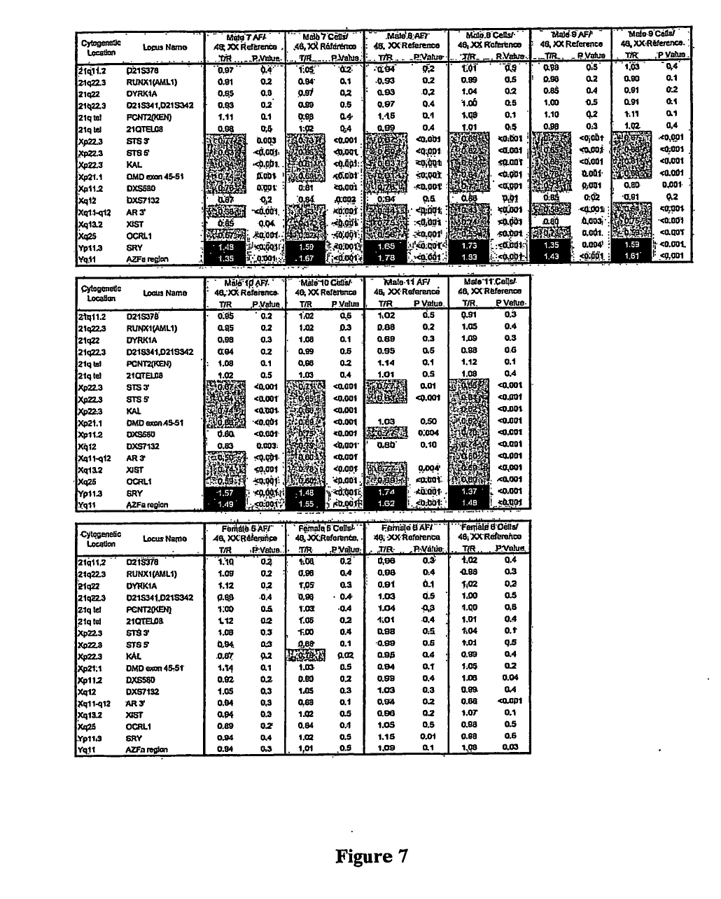

FIG. 5 shows data from two euploid and four aneuploid cell-free fetal DNA samples. For all 13 euploid fetal samples (11 others shown in FIGS. 6 and 7), markers on chromosome 21 were not significantly different from euploid reference DNA. However, the three fetal samples with trisomy 21 had increased ratios of target-to-reference intensities on most chromosome 21 markers (FIG. 5). The fetal sample with monosomy X had decreased hybridization signals on seven of nine X-chromosome markers compared with euploid female reference (FIG. 6).

FIG. 6 shows array data obtained when four euploid cell-free fetal DNA samples were hybridized separately with either male or female reference DNA. FIG. 7 shows comparison data from euploid samples in which both amniotic fluid cell-free fetal DNA and DNA from the corresponding amniocytes were hybridized to the arrays.

When the hybridization performance of cell-free fetal DNA samples was compared with samples of DNA isolated from their corresponding amniocytes, the cell-free fetal DNA and cellular DNA samples were all informative for sex, but cell-free fetal DNA samples had higher clone-clone variability (noise). Noise in the samples was assessed using the median adjacent clone ratio difference (MACRD) criterion, calculated by determining the median of the absolute Cy-3™-to-Cy-5™ fluorescent intensity ratio difference between cytogenetically adjacent clones, which should be small. Currently, the “desirable” MACRD recommended by GenoSensor analysis software for a high quality assay is <0.065 (Vysis, unpublished data). Higher MACRDs indicate poor quality hybridization, since adjacent clone pairs have similar ratios in the vast majority of cases. On average, the MACRDs for DNA isolated from amniocytes were ≦0.065, whereas cell-free fetal DNA samples exhibited values of 0.05-0.084. Although MACRDs were higher for some cell-free fetal DNA samples than for cellular DNA, in cell-free fetal DNA samples, the sensitivity of detection of chromosome-21, -X, and -Y markers, measured by normalized target/reference ratios of fluorescent intensities and P values, was similar, and quality values of array parameters, including mean intra-target coefficient of variation and modal distribution of standard deviation, were at or below the acceptable cutoffs established from multiple sets of hybridization done at Vysis for quality criteria development.

These results indicate that cell-free fetal DNA extracted from amniotic fluid can be analyzed by using CGH microarrays to correctly identify fetal sex and whole-chromosome gains or losses such as trisomy 21 and monosomy X. Cell-free fetal DNA has the advantage of being readily available from the portion of amniotic fluid that is normally discarded. Thus, it can be used in conjunction with standard karyotyping and will not interfere with the current standard of care or compromise fetal health. In addition, it does not require the time-consuming expansion of cultured cells but can be performed immediately after the specimen is received, providing a more rapid diagnosis.

In summary, molecular analysis of cell-free fetal DNA from amniotic fluid by use of CGH microarray technology is a promising technique that allows for rapid screening of samples for whole-chromosome changes, including aneuploidy, and may augment standard karyotyping techniques for pre-natal genetic diagnosis. This technology may aid the discovery and description of minor genetic aberrations, such as microdeletions and microduplications, which will potentially enhance future prenatal genetic diagnostic applications. Further investigation is warranted to explore the clinical significance of the detection of submicroscopic genetic rearrangements in the developing fetus.

What is claimed is:

1. A method of prenatal diagnosis comprising steps of:
providing a sample of amniotic fluid fetal DNA;
analyzing the amniotic fluid fetal DNA by hybridization to obtain fetal genomic information; and
based on the fetal genomic information obtained, providing a prenatal diagnosis.

2. The method of claim 1, wherein the amniotic fluid fetal DNA is obtained by:
providing a sample of amniotic fluid obtained from a woman pregnant with a fetus;
removing cell populations from the sample of amniotic fluid to obtain a remaining amniotic material; and
treating the remaining amniotic material such that cell-free fetal DNA present in the remaining material is extracted and made available for analysis, resulting in amniotic fluid fetal DNA.

3. The method of claim 2, wherein substantially all cell populations are removed from the sample of amniotic fluid and wherein the amniotic fluid fetal DNA consists essentially of cell-free fetal DNA.

4. The method of claim 2, wherein the remaining amniotic material comprises some cells and wherein the amniotic fluid fetal DNA comprises cell-free fetal DNA and DNA originating from the cells present in the remaining amniotic material.

5. The method of claim 2 further comprising steps of:
freezing the remaining amniotic material to obtain a frozen sample;
storing the frozen sample for a period of time under suitable storage conditions; and
thawing the frozen sample prior to the treating step.

6. The method of claim 5 further comprising removing substantially all cell populations that are still present in the remaining amniotic material after the thawing step and prior to the treating step.

7. The method of claim 1, wherein analyzing the amniotic fluid fetal DNA by hybridization to obtain fetal genomic information comprises using an array.

8. The method of claim 7, wherein the array is a cDNA array.

9. The method of claim 7, wherein the array is an oligonucleotide array.

10. The method of claim 7, wherein the array is a SNP array.

11. The method of claim 7, wherein analyzing the amniotic fluid fetal DNA is performed using array-based comparative genomic hybridization.

12. The method of claim 1 further comprising amplifying the amniotic fluid fetal DNA prior to the analyzing step, resulting in amplified amniotic fluid fetal DNA.

13. The method of claim 12, wherein amplifying the amniotic fluid fetal DNA comprises using PCR.

14. The method of claim 1 further comprising labeling the amniotic fluid fetal DNA with a detectable agent prior to the analyzing step, resulting in labeled amniotic fluid fetal DNA.

15. The method of claim 14, wherein the detectable agent comprises a fluorescent label.

16. The method of claim 15, wherein the fluorescent label comprises a fluorescent dye selected from the group consisting of Cy-3™, Cy-5™, Texas Red, FITC, Spectrum Red™, Spectrum Green™, phycoerythrin, a rhodamine, a fluorescein, a fluorescein isothiocyanate, a carbocyanine, a merocyanine, a styryl dye, an oxonol dye, a BODIPY dye, equivalents thereof, analogues thereof, derivatives thereof, and any combination thereof.

17. The method of claim 15, wherein the fluorescent label comprises Cy-3™ or Cy-5™.

18. The method of claim 15, wherein the fluorescent label comprises Spectrum Red™ or Spectrum Green™.

19. The method of claim 14, wherein labeling the amniotic fluid fetal DNA comprises random priming, nick translation, PCR or tailing.

20. The method of claim 14, wherein the detectable agent comprises biotin or dioxigenin.

21. The method of claim 1, wherein fetal genomic information includes chromosomal abnormalities and genome copy number changes at multiple genomic loci.

22. The method of claim 1, wherein providing a prenatal diagnosis comprises determining the sex of the fetus.

23. The method of claim 1, wherein providing a prenatal diagnosis comprises detecting and identifying a chromosomal abnormality.

24. The method of claim 1, wherein providing a prenatal diagnosis comprises identifying a disease or condition associated with a chromosomal abnormality.

25. The method of claim 2, wherein the fetus is suspected of having a chromosomal abnormality.

26. The method of claim 2, wherein the fetus is suspected of having a disease or condition associated with a chromosomal abnormality.

27. The method of claim 2, wherein the pregnant woman is 35 or more than 35 years old.

28. The method of claim 23, 24, 25 or 26, wherein the chromosomal abnormality is selected from the group consisting of an extra individual chromosome, a missing individual chromosome, an extra portion of a chromosome, a missing portion of a chromosome, a break, a ring, a chromosomal rearrangement, and any combination thereof.

29. The method of claim 23, 24, 25 or 26, wherein the chromosomal abnormality is a chromosomal rearrangement selected from the group consisting of a translocation, an inversion, a duplication, a deletion, an addition, and any combination thereof.

30. The method of claim 23, 24, 25 or 26, wherein the chromosomal abnormality is selected from the group consisting of an extra chromosome 21, a missing chromosome 21, an extra portion of chromosome 21, a missing portion of chromosome 21, a rearrangement of chromosome 21, and any combination thereof.

31. The method of claim 23, 24, 25 or 26, wherein the chromosomal abnormality is not detectable by G-banding analysis or metaphase CGH.

32. The method of claim 23, 24, 25 or 26, wherein the chromosomal abnormality is a microdeletion, a microduplication, or a subtelomeric rearrangement.

33. The method of claim 23, 24, 25 or 26, wherein the chromosomal abnormality is selected from the group consisting of an extra chromosome 13, 18, X or Y, a chromosomal aberration involving chromosome 1, a deletion of chromosome portion 1q21, a deletion of chromosome portion 4p16, a chromosomal aberration involving chromosome 4, a deletion on chromosome 5, a chromosomal aberration involving chromosome 7, a deletion of chromosome portion 7q11.23, a chromosomal aberration involving chromosome 8, a translocation involving chromosome 9 and chromosome 22, a chromosomal aberration involving chromosome 10, a chromosomal aberration involving chromosome 11, a deletion of chromosome portion 13q14, a deletion of chromosome portion 15q11-q13, a deletion of chromosome portion 15q21.1, a deletion of chromosome portion 16p13.3, a deletion of chromosome portion 17p11.2, a deletion of chromosome portion 17p13.3, a chromosomal aberration involving chromosome 19, a deletion of chromosome portion 22q11, and a chromosomal aberration involving chromosome X.

34. The method of claim 24 or 26, wherein the disease or condition associated with a chromosomal abnormality is an aneuploidy.

35. The method of claim 34, wherein the aneuploidy is selected from the group consisting of Down syndrome, Patau syndrome, Edward syndrome, Turner syndrome, Klinefelter syndrome and XYY disease.

36. The method of claim 24 or 26, wherein the disease or condition associated with a chromosomal abnormality is an X-linked disorder.

37. The method of claim 36, wherein the X-linked disorder is selected from the group consisting of Hemophilia A, Duchenne muscular dystrophy, Lesch-Nyhan syndrome, severe combined immunodeficiency, and Fragile X syndrome.

38. The method of claim 24 or 26, wherein the disease or condition is associated with a chromosomal abnormality that is not detectable by G-banding analysis or metaphase CGH.

39. The method of claim 24 or 26, wherein the disease or condition associated with a chromosomal abnormality is a microdeletion/microduplication syndrome.

40. The method of claim 39, wherein the microdeletion/microduplication syndrome is selected from the group consisting of Prader-Willi syndrome, Angelman syndrome, DiGeorge syndrome, Smith-Magenis syndrome, Rubinstein-Taybi syndrome, Miller-Dieker syndrome, Williams syndrome, and Charcot-Marie-Tooth syndrome.

41. The method of claim 24 or 26, wherein the disease or condition is associated with a subtelomeric rearrangement.

42. The method of claim 24 or 26, wherein the disease or condition associated with a chromosomal abnormality is selected from the group consisting of Cri du Chat syndrome, Retinoblastoma, Wolf-Hirschhorn syndrome, Wilms tumor, spinobulbar muscular atrophy, cystic fibrosis, Gaucher disease, Marfan syndrome, and sickle cell anemia.

43. A method of prenatal diagnosis performed by analyzing amniotic fluid fetal DNA by array-based comparative genomic hybridization, the method comprising steps of:
providing a test sample of amniotic fluid fetal DNA, wherein the test sample comprises a plurality of nucleic acid segments comprising a substantially complete first genome with an unknown karyotype and labeled with a first detectable agent;
providing a reference sample, wherein the reference sample comprises a plurality of nucleic acid segments comprising a substantially complete second genome with a known karyotype and labeled with a second detectable agent;
providing an array comprising a plurality of genetic probes, wherein each genetic probe is immobilized to a discrete spot on a substrate surface to form the array and wherein together the genetic probes comprise a substantially complete third genome or a subset of a third genome;
contacting the array simultaneously with the test and reference samples under conditions wherein the nucleic acid segments in the samples can specifically hybridize to the genetic probes on the array;
determining the binding of the individual nucleic acids of the test sample and reference sample to the individual genetic probes immobilized on the array to obtain a relative binding pattern; and
based on the relative binding pattern obtained, providing a prenatal diagnosis.

44. The method of claim 43, wherein the nucleic acids of the test sample and reference sample are labeled by random priming, nick translation, PCR or tailing.

45. The method of claim 43, wherein the first detectable agent comprises a first fluorescent label and the second detectable agent comprises a second fluorescent label.

46. The method of claim 43, wherein the first fluorescent label and second fluorescent label produce a dual-color fluorescence upon excitation.

47. The method of claim 46, wherein the first fluorescent label comprises Cy-3™ and the second fluorescent label comprises Cy-5™.

48. The method of claim 46, wherein the first fluorescent label comprises Cy-5™ and the second fluorescent label comprises Cy-3™.

49. The method of claim 46, wherein the first fluorescent label comprises Spectrum Red™ and the second fluorescent label comprises Spectrum Green™.

50. The method of claim 46, wherein the first fluorescent label comprises Spectrum Green™ and the second fluorescent label comprises Spectrum Red™.

51. The method of claim 43, wherein the hybridization capacity of high copy number repeat sequences present in the nucleic acid segments of the test sample and reference sample is suppressed.

52. The method of claim 51, wherein the hybridization capacity of high copy number repeat sequences is suppressed by adding unlabeled blocking nucleic acids to the test sample and reference sample prior to the contacting step.

53. The method of claim 52, wherein the unlabeled blocking nucleic acids are Human Cot-1 DNA.

54. The method of claim 43, wherein the amniotic fluid fetal DNA is obtained by:
providing a sample of amniotic fluid obtained from a woman pregnant with a fetus;
removing cell populations from the sample of amniotic fluid to obtain a remaining amniotic material; and
treating the remaining amniotic material such that cell-free fetal DNA present in the remaining material is extracted and made available for analysis, resulting in amniotic fluid fetal DNA.

55. The method of claim 54, wherein substantially all cell populations are removed from the sample of amniotic fluid and wherein the amniotic fluid fetal DNA consists essentially of cell-free fetal DNA.

56. The method of claim 54, wherein the remaining amniotic material comprises some cells and wherein the amniotic fluid fetal DNA comprises cell-free fetal DNA and DNA originating from the cells present in the remaining amniotic material.

57. The method of claim 54 further comprising steps of:
freezing the remaining amniotic material to obtain a frozen sample;
storing the frozen sample for a period of time under suitable storage conditions; and
thawing the frozen sample prior to the treating step.

58. The method of claim 54 further comprising amplifying the amniotic fluid fetal DNA using PCR, resulting in amplified amniotic fluid fetal DNA.

59. The method of claim 54 further comprising labeling the amniotic fluid fetal DNA with a detectable agent by random priming, nick translation, PCR or tailing, resulting in labeled amniotic fluid fetal DNA.

60. The method of claim 43, wherein the karyotype of the second genome has been determined by G-banding analysis, metaphase CGH, FISH or SKY.

61. The method of claim 43, wherein determining the binding of the individual nucleic acids of the test and reference samples to the individual genetic probes immobilized on the array to obtain a relative binding pattern comprises steps of:
measuring the intensity of the signals produced by the first detectable agent and second detectable agent at each discrete spot on the array; and
determining the ratio of the intensities of the signals for each spot of the array.

62. The method of claim 43, wherein determining the binding of the individual nucleic acids of the test and reference samples to the individual genetic probes immobilized on the array to obtain a relative binding pattern comprises steps of:
using a computer-assisted imaging system capable of acquiring multicolor fluorescence images to obtain a fluorescence image of the array after hybridization; and
using a computer-assisted image analysis system to analyze the fluorescence image obtained, to interpret data imaged from the array and to display results as genome copy number ratios as a function of genomic locus in the third genome.

63. The method of claim 43, wherein providing a prenatal diagnosis comprises determining the sex of the fetus carried by the pregnant woman.

64. The method of claim 43, wherein providing a prenatal diagnosis comprises detecting and identifying a chromosomal abnormality.

65. The method of claim 43, wherein providing a prenatal diagnosis comprises identifying a disease or condition associated with a chromosomal abnormality.

66. The method of claim 43, wherein the amniotic fluid fetal DNA originates from a fetus suspected of having a chromosomal abnormality.

67. The method of claim 43, wherein the amniotic fluid fetal DNA originates from a fetus suspected of having a disease or condition associated with a chromosomal abnormality.

68. The method of claim 43, wherein the amniotic fluid fetal DNA has been extracted from a sample of amniotic fluid obtained from a pregnant woman who is 35 or more than 35 years old.

69. The method of claim 64, 65, 66 or 67, wherein the chromosomal abnormality is selected from the group consisting of an extra individual chromosome, a missing individual chromosome, an extra portion of a chromosome, a missing portion of a chromosome, a break, a ring, a chromosomal rearrangement, and any combination thereof.

70. The method of claim 64, 65, 66 or 67, wherein the chromosomal abnormality is a chromosomal rearrangement selected from the group consisting of a translocation, an inversion, a duplication, a deletion, an addition, and any combination thereof.

71. The method of claim 64, 65, 66 or 67, wherein the chromosomal abnormality is selected from the group consisting of an extra chromosome 21, a missing chromosome 21, an extra portion of chromosome 21, a missing portion of chromosome 21, a rearrangement of chromosome 21, and any combination thereof.

72. The method of claim 64, 65, 66 or 67, wherein the chromosomal abnormality is not detectable by G-banding analysis or metaphase CGH.

73. The method of claim 64, 65, 66 or 67, wherein the chromosomal abnormality is a microdeletion, a microduplication or a subtelomeric rearrangement.

74. The method of claim 64, 65, 66 or 67, wherein the chromosomal abnormality is selected from the group consisting of an extra chromosome 13, 18, X or Y, a chromosomal aberration involving chromosome 1, a deletion of chromosome portion 1q21, a deletion of chromosome portion 4p16, a chromosomal aberration involving chromosome 4, a deletion on chromosome 5, a chromosomal aberration involving chromosome 7, a deletion of chromosome portion 7q11.23, a chromosomal aberration involving chromosome 8, a translocation involving chromosome 9 and chromosome 22, a chromosomal aberration involving chromosome 10, a chromosomal aberration involving chromosome 11, a deletion of chromosome portion 13q14, a deletion of chromosome portion 15q11-q13, a deletion of chromosome portion 15q21.1, a deletion of chromosome portion 16p13.3, a deletion of chromosome portion 17p 11.2, a deletion of chromosome portion 17p13.3, a chromosomal aberration involving chromosome 19, a deletion of chromosome portion 22q11, and a chromosomal aberration involving chromosome X.

75. The method of claim 65 or 67, wherein the disease or condition associated with a chromosomal abnormality is an aneuploidy.

76. The method of claim 75, wherein the aneuploidy is selected from the group consisting of Down syndrome, Patau syndrome, Edward syndrome, Turner syndrome, Klinefelter syndrome and XYY disease.

77. The method of claim 65 or 67, wherein the disease or condition associated with a chromosomal abnormality is an X-linked disorder.

78. The method of claim 77, wherein the X-linked disorder is selected from the group consisting of Hemophilia A, Duchenne muscular dystrophy, Lesch-Nyhan syndrome, severe combined immunodeficiency, and Fragile X syndrome.

79. The method of claim 65 or 67, wherein the disease or condition is associated with a chromosomal abnormality that is not detectable by G-banding analysis or metaphase CGH.

80. The method of claim 65 or 67, wherein the disease or condition associated with a chromosomal abnormality is a microdeletion/microduplication syndrome.

81. The method of claim 80, wherein the microdeletion/microduplication syndrome is selected from the group consisting of Prader-Willi syndrome, Angelman syndrome, DiGeorge syndrome, Smith-Magenis syndrome, Rubinstein-Taybi syndrome, Miller-Dieker syndrome, Williams syndrome, and Charcot-Marie-Tooth syndrome.

82. The method of claim 65 or 67, wherein the disease or condition is associated with a subtelomeric rearrangement.

83. The method of claim 65 or 67, wherein the disease or condition associated with a chromosomal abnormality is selected from the group consisting of Cri du Chat syndrome, Retinoblastoma, Wolf-Hirschhorn syndrome, Wilms tumor, spinobulbar muscular atrophy, cystic fibrosis, Gaucher disease, Marfan syndrome, and sickle cell anemia.

84. A method of testing amniotic fluid fetal DNA by array-based comparative genomic hybridization comprising steps of:
providing a test sample of amniotic fluid fetal DNA, wherein the test sample comprises a plurality of nucleic acid segments comprising a substantially complete first genome with a chromosomal micro-abnormality and labeled with a first detectable agent;
providing a reference sample of control genomic DNA, wherein the reference sample comprises a plurality of nucleic acid segments comprising a substantially complete second genome with a known karyotype and labeled with a second detectable agent;
providing an array comprising a plurality of genetic probes, wherein each genetic probe is immobilized to a discrete spot on a substrate surface to form the array and wherein together the genetic probes comprise a substantially complete third genome or a subset of a third genome;
contacting the array simultaneously with the test sample and reference sample under conditions wherein the nucleic acid segments of the test and reference samples can specifically hybridize to the genetic probes immobilized on the array;
using a computer-assisted imaging system capable of acquiring multicolor fluorescence images to obtain a fluorescence image of the array after hybridization;
using a computer-assisted image analysis system to analyze the fluorescence image obtained, to interpret data imaged from the array and to display results as genome copy number ratios as a function of genomic locus in the third genome;
determining the karyotype of the first genome by FISH analysis; and
comparing the results displayed as genome copy number ratios to the karyotype of the first genome determined by FISH.

85. The method of claim 84, wherein comparing the results displayed as genome copy number ratios to the karyotype of the first genome determined by FISH comprises evaluating the degree of consistency between the results displayed and the karyotype of the first genome determined by FISH.

86. The method of claim 84, wherein comparing the results displayed as genome copy number ratios to the karyotype of the first genome determined by FISH comprises comparing the sensitivity of detection of the chromosomal micro-abnormality present in the first genome by FISH and by array-based comparative genomic hybridization.

87. The method of claim 84, wherein comparing the results displayed as genome copy number ratios to the karyotype of the first genome determined by FISH comprises comparing the selectivity of detection of the chromosomal micro-abnormality present in the first genome by FISH and by array-based comparative genomic hybridization.

88. The method of claim 84, wherein the chromosomal micro-abnormality is a microdeletion, a microduplication or a subtelomeric rearrangement.

89. The method of claim 84, wherein the chromosomal micro-abnormality is selected from the group consisting of a deletion of chromosome portion 1q22, a deletion of chromosome portion 7q11.23, a deletion of chromosome portion 8q21, a deletion of chromosome portion 10q21.1-q22.1, a deletion of chromosome portion 15q11-q13, a deletion of chromosome portion 16p13.3, a deletion of chromosome portion 17p11.2, a deletion of chromosome portion 17p13.3, a deletion of chromosome portion 19q13.1-q13.2, and a deletion of chromosome portion 22q11.2.

90. The method of claim 84, wherein the nucleic acids of the test sample and reference sample are labeled by random priming, nick translation, PCR or tailing.

91. The method of claim 84, wherein the first detectable agent comprises a first fluorescent label, the second detectable agent comprises a second fluorescent label, and the first and second fluorescent labels produce a dual-color fluorescence upon excitation.

92. The method of claim 91, wherein the first fluorescent label comprises Cy-3™ and the second fluorescent label comprises Cy-5™.

93. The method of claim 91, wherein the first fluorescent label comprises Cy-3™ and the second fluorescent label comprises Cy-3™.

94. The method of claim 91, wherein the first fluorescent label comprises Spectrum Red™ and the second fluorescent label comprises Spectrum Green™.

95. The method of claim 91, wherein the first fluorescent label comprises Spectrum Green™ and the second fluorescent label comprises Spectrum Red™.

96. The method of claim 84, wherein the hybridization capacity of high copy number repeat sequences present in the nucleic acid segments of the test sample and reference sample is suppressed by adding Human Cot-1 DNA to the test and reference samples before the contacting step.

97. The method of claim 84, wherein the amniotic fluid fetal DNA is obtained by:
providing a sample of amniotic fluid obtained from a woman pregnant with a fetus;
removing cell populations from the sample of amniotic fluid to obtain a remaining amniotic material; and
treating the remaining amniotic material such that cell-free fetal DNA present in the remaining material is extracted and made available for analysis, resulting in amniotic fluid fetal DNA.

98. The method of claim 97, wherein substantially all cell populations are removed from the sample of amniotic fluid and wherein the amniotic fluid fetal DNA consists essentially of cell-free fetal DNA.

99. The method of claim 97, wherein the remaining amniotic material comprises some cells and wherein the amniotic fluid fetal DNA comprises cell-free fetal DNA and DNA originating from the cells present in the remaining amniotic material.

100. The method of claim 97 further comprising steps of:
freezing the remaining amniotic material to obtain a frozen sample;
storing the frozen sample for a period of time under suitable storage conditions; and
thawing the frozen sample prior to the treating step.

101. The method of claim 97 further comprising amplifying the amniotic fluid fetal DNA using PCR, resulting in amplified amniotic fluid fetal DNA.

102. The method of claim 97 further comprising labeling the amniotic fluid fetal DNA with a detectable agent by random priming, nick translation, PCR or tailing, resulting in labeled extracted amniotic fluid fetal DNA.

103. The method of claim 84, wherein the karyotype of the second genome has been determined by G-banding analysis, metaphase CGH, FISH or SKY.

104. A method for identifying a chromosomal abnormality by analyzing amniotic fluid fetal DNA by array-based comparative genomic hybridization, the method comprising steps of:
providing a test sample of amniotic fluid fetal DNA, wherein the amniotic fluid fetal DNA originates from a fetus determined to have multiple congenital anomalies by sonographic examination, and wherein the test sample comprises a plurality of nucleic acid segments comprising a substantially complete first genome with a normal karyotype and labeled with a first detectable agent;
providing a reference sample of control amniotic fluid fetal DNA, wherein the control amniotic fluid fetal DNA originates from a fetus determined to have no congenital anomalies by sonographic examination, and wherein the reference sample comprises a plurality of nucleic acid segments comprising a substantially complete second genome with a normal karyotype and labeled with a second detectable agent;
providing an array comprising a plurality of genetic probes, wherein each genetic probe is immobilized to a discrete spot on a substrate surface to form the array and wherein together the genetic probes comprise a substantially complete third genome or a subset of a third genome;
contacting the array simultaneously with the test sample and reference sample under conditions wherein the nucleic acid segments in the samples can specifically hybridize to the genetic probes immobilized on the array;
using a computer-assisted imaging system capable of acquiring multicolor fluorescence images to obtain a fluorescence image of tie array after hybridization;
using a computer-assisted image analysis system to analyze the fluorescence image obtained, to interpret data imaged from the array and to display results as genome copy number ratios as a function of genomic locus in the third genome; and
analyzing the results displayed to detect and identify any chromosomal abnormality present.

105. The method of claim 104, wherein the karyotype of the test sample has been determined by metaphase CGH analysis with a 550 band level of resolution.

106. The method of claim 104, wherein the chromosomal abnormality present in the first genome is a chromosomal micro-abnormality that is not detectable by metaphase CGH analysis with a 550 band level of resolution.

107. The method of claim 106, wherein the chromosomal micro-abnormality is selected from the group consisting of a micro-addition, a micro-deletion, a micro-duplication, a micro-inversion, a micro-translocation, a subtelomeric rearrangement and any combination thereof.

108. The method of claim 104, wherein the nucleic acids of the test sample and reference sample are labeled by random priming, nick translation, PCR or tailing.

109. The method of claim 104, wherein the first detectable agent comprises a first fluorescent label, the second detectable agent comprises a second fluorescent label, and the first and second fluorescent labels produce a dual-color fluorescence upon excitation.

110. The method of claim 109, wherein the first fluorescent label comprises Cy-3™ and the second fluorescent label comprises Cy-5™.

111. The method of claim 109, wherein the first fluorescent label comprises Cy-5™ and the second fluorescent label comprises Cy-3™.

112. The method of claim 109, wherein the first fluorescent label comprises Spectrum Red™ and the second fluorescent label comprises Spectrum Green™.

113. The method of claim 109, wherein the first fluorescent label comprises Spectrum Green™ and the second fluorescent label comprises Spectrum Red™.

114. The method of claim 104, wherein the hybridization capacity of high copy number repeat sequences present in the nucleic acid segments of the test sample and reference sample is suppressed by adding Human Cot-1 DNA to the test and reference samples before the contacting step.

115. The method of claim 104, wherein the amniotic fluid fetal DNA from the test sample is obtained by:
providing a sample of amniotic fluid obtained from a woman pregnant with a fetus;
removing cell populations from the sample of amniotic fluid to obtain a remaining amniotic material; and
treating the remaining amniotic material such that cell-free fetal DNA present in the remaining material is extracted and made available for analysis, resulting in amniotic fluid fetal DNA.

116. The method of claim 115, wherein substantially all cell populations are removed from the sample of amniotic fluid and the amniotic fluid fetal DNA consists essentially of cell-free fetal DNA.

117. The method of claim 115, wherein the remaining amniotic material comprises some cells and the amniotic fluid fetal DNA comprises cell-free fetal DNA and DNA originating from the cells present in the remaining amniotic material.

118. The method of claim 104, wherein the control amniotic fluid fetal DNA from the reference sample is obtained by:
providing a sample of amniotic fluid obtained from a woman pregnant with a fetus;
removing cell populations from the sample of amniotic fluid to obtain a remaining amniotic material; and
treating the remaining amniotic material such that cell-free fetal DNA present in the remaining material is extracted and made available for analysis, resulting in control amniotic fluid fetal DNA.

119. The method of claim 118, wherein substantially all cell populations are removed from the sample of amniotic fluid and the control amniotic fluid fetal DNA consists essentially of cell-free fetal DNA.

120. The method of claim 118, wherein the remaining amniotic material comprises some cells and the control amniotic fluid fetal DNA comprises cell-free fetal DNA and DNA originating from the cells present in the remaining amniotic material.

121. The method of claim 115 or 118 further comprising steps of:
freezing the remaining amniotic material to obtain a frozen sample;
storing the frozen sample for a period of time under suitable storage conditions; and
thawing the frozen sample prior to the treating step.

122. The method of claim 115 further comprising amplifying the amniotic fluid fetal DNA using PCR, resulting in amplified amniotic fluid fetal DNA.

123. The method of claim 118 further comprising amplifying the control amniotic fluid fetal DNA using PCR, resulting in amplified control amniotic fluid fetal DNA

124. The method of claim 115 further comprising labeling the amniotic fluid fetal DNA with a detectable agent by random priming, nick translation, PCR or tailing, resulting in labeled amniotic fluid fetal DNA.

125. The method of claim 118 further comprising labeling the control amniotic fluid fetal DNA with a detectable agent by random priming, nick translation, PCR or tailing, resulting in labeled control amniotic fluid fetal DNA.

126. The method of claim 104, wherein the karyotype of the second genome has been determined by G-banding analysis, metaphase CGH, FISH or SKY.

127. The method of claim 104, wherein the test and reference samples are matched for fetal gender, site of sample acquisition, gestational age, and storage time.

128. A kit comprising the following components:
materials to extract cell-free fetal DNA from a sample of amniotic fluid obtained from a pregnant woman;
an array comprising a plurality of genetic probes, wherein each genetic probe is immobilized to a discrete spot on a substrate surface to form the array and wherein together the genetic probes comprise a substantially complete genome or a subset of a genome; and
instructions for using the array as set forth in claim 43, 84 or 104.

129. The kit of claim 128 further comprising materials to label a first sample of DNA with a first detectable agent and a second sample of DNA with a second detectable agent.

130. The kit of claim 129, wherein the first detectable agent comprises a first fluorescent label, the second detectable agent comprises a second fluorescent label, and the first and second fluorescent labels produce a dual-color fluorescence upon excitation.

131. The kit of claim 130 further comprising materials to label a first sample of DNA and a second sample of DNA with Cy-3™ and Cy-5™.

132. The kit of claim 130 further comprising materials to label a first sample of DNA and a second sample of DNA with Spectrum Red™ and Spectrum Green™.

133. The kit of claim 128 further comprising a sample of control genomic DNA with a normal, female karyotype.

134. The kit of claim 128 further comprising a sample of control genomic DNA with a normal, male karyotype.

135. The kit of claim 128 further comprising a sample of control genomic DNA with a karyotype comprising a chromosomal abnormality.

136. The kit of claim 128 further comprising hybridization and wash buffers.

137. The kit of claim 128 further comprising Human Cot-1 DNA.
